SEQUENCE LISTING (16 sequences)

SEQ ID NO: 1 - DNA, 24 bp, Artificial Sequence
  synthetic
tgctggcctaatagagtggcaaag

SEQ ID NO: 2 - DNA, 21 bp, Artificial Sequence
  synthetic
ggcatgtcccactatcactgt

SEQ ID NO: 3 - DNA, 20 bp, Artificial Sequence
  synthetic
aatcatgaaagtcgccagtg

SEQ ID NO: 4 - DNA, 21 bp, Artificial Sequence
  synthetic
atgtcgtagagcagcacgttt

SEQ ID NO: 5 - DNA, 21 bp, Artificial Sequence
  synthetic
cagcaggtccatgtggctact

SEQ ID NO: 6 - DNA, 19 bp, Artificial Sequence
  synthetic
gccgtttccgtttctttcc

SEQ ID NO: 7 - DNA, 21 bp, Artificial Sequence
  synthetic
gatgcttatagggcggagtgg

SEQ ID NO: 8 - DNA, 21 bp, Artificial Sequence
  synthetic
gctgaacgaggtctttttggt

SEQ ID NO: 9 - DNA, 21 bp, Artificial Sequence
  synthetic
tttctgggtgacggtgatctc

SEQ ID NO: 10 - DNA, 22 bp, Artificial Sequence
  synthetic
catatgtccaatcccagtgcaa

SEQ ID NO: 11 - DNA, 23 bp, Artificial Sequence
  synthetic
catgagaagtatgacaacagcct

SEQ ID NO: 12 - DNA, 22 bp, Artificial Sequence
  synthetic
agtccttccacgataccaaagt

SEQ ID NO: 13 - DNA, 20 bp, Artificial Sequence
  synthetic
cctgcgactccttgacgttg

SEQ ID NO: 14 - DNA, 21 bp, Artificial Sequence
  synthetic
agcggtgaaagtggtttggtt

SEQ ID NO: 15 - DNA, 25 bp, Artificial Sequence
  synthetic
tcacccacactgtgcccatctacga

SEQ ID NO: 16 - DNA, 25 bp, Artificial Sequence
  synthetic
cagcggaaccgctcattgccaatgg

(19) United States
(12) Patent Application Publication
(10) Pub. No.: US 2022/0332840 A1
(43) Pub. Date: Oct. 20, 2022

(54) SENSITIZATION OF TUMORS TO THERAPIES THROUGH ENDOGLIN ANTAGONISM

(71) Applicant: Cedars-Sinai Medical Center, Los Angeles, CA (US)

(72) Inventors: Neil Bhowmick, Beverly Hills, CA (US); Bethany Smith, Los Angeles, CA (US); Veronica Placencio, Valley Village, CA (US); Anisha Madhav, Woodland, CA (US)

(73) Assignee: Cedars-Sinai Medical Center, Los Angeles, CA (US)

(21) Appl. No.: 17/685,040

(22) Filed: Mar. 2, 2022

Related U.S. Application Data
(63) Continuation of application No. 16/305,821, filed on Nov. 29, 2018, now abandoned.
(60) Provisional application No. 62/350,017, filed on Jun. 14, 2016.

(51) Int. Cl. C07K 16/28; A61P 35/00

(52) U.S. Cl. CPC C07K 16/2896; A61P 35/00; A61K 2039/505

(57) ABSTRACT

Described herein is a method of sensitizing a cancer in a subject and methods of treating, slowing the progression of, reducing the severity of, preventing the recurrence of, and/or reducing the recurrence likelihood of a cancer in a subject. The invention further provides for a method of preventing the recurrence of and/or reducing the recurrence likelihood of a cancer in a subject who has been treated with a cancer therapy.

### STATEMENT REGARDING FEDERALLY-SPONSORED RESEARCH

This invention was made with government support under Grant No. CA108646 awarded by the National Institutes of Health. The government has certain rights in the invention.

### FIELD OF THE INVENTION

The invention relates to medicine and cancer.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Endoglin (also referred as CD105) was originally identified as a receptor expressed on proliferating endothelial cells and consequential to the survival of blood vessels. An endoglin antagonist (i.e., TRC105 from Tracon Pharmaceuticals Inc.) was hence developed for the purpose of killing tumors especially dependent on new vasculature.

In this invention, we provide methods, kits and systems for treating cancers and tumors through combining CD105 antagonists and various treatments including but not limited to chemotherapy, radiation therapy, hormone therapy and surgeries.

### SUMMARY

The following embodiments and aspects thereof are described and illustrated in conjunction with systems, compositions and methods which are meant to be exemplary and illustrative, not limiting in scope.

Various embodiments of the present invention provide for a method of sensitizing a cancer in a subject in need thereof, comprising: providing a CD105 antagonist; and administering the CD105 antagonist to the subject, thereby sensitizing the cancer. In various embodiments, the method further comprises administering a cancer therapy. In various embodiments, the method further comprises identifying a subject in need of sensitizing a cancer to cancer treatment before administering the CD105 antagonist.

In various embodiments, the cancer is prostate cancer, breast cancer, bladder cancer, lung cancer, colorectal cancer, pancreatic cancer, liver cancer, renal cancer, renal cell carcinoma, melanoma, sarcoma, head and neck cancer, glioblastoma, or a combination thereof. In various embodiments, the cancer is resistant to radiation and/or androgen targeted therapy. In various embodiments, the cancer is prostate cancer.

In various embodiments, the CD105 antagonist is an antibody specifically binding to CD105 or an antigen-binding fragment thereof. In various other embodiments, the CD105 antagonist is TRC105 or an antigen-binding fragment thereof.

In various embodiments, the cancer therapy is radiotherapy, chemotherapy, hormone therapy, or surgery, or a combination thereof. In various embodiments, the subject is treated by the administration of the CD105 antagonist and the cancer therapy.

Various embodiments of the present invention provide for a method of treating, slowing the progression of, reducing the severity of, preventing the recurrence of, and/or reducing the recurrence likelihood of a cancer in a subject in need thereof, comprising: administering a CD105 antagonist to the subject; and administering a cancer therapy to the subject, thereby treating, slowing the progression of, reducing the severity of, preventing the recurrence of, and/or reducing the recurrence likelihood of the cancer in the subject.

In various embodiments, the cancer is prostate cancer, breast cancer, bladder cancer, lung cancer, colorectal cancer, pancreatic cancer, liver cancer, renal cancer, renal cell carcinoma, melanoma, sarcoma, head and neck cancer, glioblastoma, or a combination thereof. In various embodiments, the cancer is resistant to radiation and/or androgen targeted therapy. In various other embodiments, the cancer is prostate cancer.

In various embodiments, the CD105 antagonist is an antibody specifically binding to CD105 or an antigen-binding fragment thereof. In various other embodiments, the CD105 antagonist is TRC105 or an antigen-binding fragment thereof.

In various embodiments, the cancer therapy is radiotherapy, chemotherapy, hormone therapy, or surgery, or a combination thereof.

Various embodiments of the present invention provide for a method of preventing the recurrence of and/or reducing the recurrence likelihood of a cancer in a subject who has been treated with a cancer therapy, comprising: administering a CD105 antagonist to the subject; and administering a subsequent cancer therapy, thereby preventing the recurrence of and/or reducing the recurrence likelihood of the cancer.

In various embodiments, the cancer is prostate cancer, breast cancer, bladder cancer, lung cancer, colorectal cancer, pancreatic cancer, liver cancer, renal cancer, renal cell carcinoma, melanoma, sarcoma, head and neck cancer, glioblastoma, or a combination thereof. In various embodiments, the cancer is resistant to radiation and/or androgen targeted therapy. In various embodiments, the cancer is prostate cancer.

In various embodiments, the CD105 antagonist is an antibody specifically binding to CD105 or an antigen-binding fragment thereof. In various embodiments, the CD105 antagonist is TRC105 or an antigen-binding fragment thereof.

In various embodiments, the subsequent cancer therapy is radiotherapy, chemotherapy, hormone therapy, or surgery, or a combination thereof.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with various embodiments of the invention, an example of the role stromal regulation plays in tumor progression.

FIG. 2 depict, in accordance with various embodiments of the invention, that androgen ablation therapy can promote CD105 expression in stromal and epithelial compartments.) Human prostate cancer (PCa) epithelial cells were grown in 3D co-cultures with mouse fibroblasts under hypoxia (2% O₂) with the indicated treatments. After 72 hours, the cells were dissociated and assessed by FACS for CD105 expression as shown. Antagonizing CD105 by either M1043 (a monoclonal rat anti-mouse CD105 antibody) or TRC105 down regulated enzalutamide-induced CD105 cell surface expression in both mouse prostatic fibroblasts and human prostate cancer epithelia.

FIG. 3 depicts, in accordance with various embodiments of the invention, that androgen receptor variants are up regulated by androgen deprivation therapy.

FIG. 4 depicts, in accordance with various embodiments of the invention, that androgen receptor variants and RNPC1 (also known as RBM38) are down regulated by TRC105 Enzalutamide up regulates RNPC1 expression.

FIG. 5 depicts, in accordance with various embodiments of the invention, that androgen receptor variants are down-regulated by TRC105 in a RNPC1 dependent manner. RNPC1 expression is elevated in prostate cancer epithelia and stromal cells.

FIG. 6 depicts, in accordance with various embodiments of the invention, TRC105 dosage response in CW22Rv1 cells.

FIG. 7 depicts, in accordance with various embodiments of the invention, that M1043 (a mouse-specific CD105 neutralizing antibody used as an antagonist) combination treatment with enzalutamide does not reduce prostate tumor xenografts. Tissue recombinant human CW22Rv1/CAF orthotropic xenografts had reduced vascularization.

FIGS. 8A-8B depict, in accordance with various embodiments of the invention, that TRC105 serves as a radiation sensitizer for prostate cancer cells. FIG. 8A) Cell cycle analysis demonstrate a chronic up regulation of the G2-phase (associated with DNA replication) when radiation is combined with TRC105 in human prostate epithelial cell line CW22Rv1. Within each group, the left column depicts G1, middle column depicts S and the right column depicts G2. FIG. 8B) CW22Rv1, prostatic epithelia, has a precipitous down regulation of survival proteins (survivin and full length PARP1) upon 4Gy radiation and TRC105 treatment. All studies shown are 5 days after irradiation and/or 5 days of treatment with TRC105.

FIG. 9 depicts, in accordance with various embodiments of the invention, that TRC105 serves as a taxane sensitizer for prostate cancer cells. The PC3 cells used in the cell death assay were treated with different concentrations of docetaxel in the presence of different concentrations of TRC105.

FIGS. 10A-10D depict that stromal heterogeneity is necessary for tumor promoting capacity, in accordance with various embodiments of the invention. FIG. 10A) Pie charts illustrate the relative ratio of the indicated stromal fibroblastic populations based on cell surface expression of the indicated markers, n>3. FIG. 10B) Scatter plot indicates tumor volume for tissue recombinant tumors made up of indicated fibroblastic populations and CW22Rv1. The bar indicates tumor volume, n>4. FIG. 10C) Histology for representative recombinant tumor sections of Rv1 with the indicated fibroblastic populations. H&E staining shows tumor morphology (scale bar represents 64 μm). Ki67 and survivin immune-localization, with hematoxylin nuclear counterstain (scale bar represents 32 μm), is quantitated, n>5. FIG. 10D) Of the top 200 differentially expressed genes identified by RNA sequencing 33 coded for secreted proteins. Venn diagram illustrates the distribution of the secreted genes annotated in the heat map according to indicated log transformed gene expression. The lines above the heat map correspond to the genes found within the groups of the Venn diagram. One-way ANOVA and Bonferroni post hoc correction was performed, error bars are mean+/−SD, and *p<0.05, **p<0.01, ****p<0.0001.

FIGS. 11A-11E depict that stromal CD105 expression is associated with NED of the adjacent epithelia, in accordance with various embodiments of the invention. FIG. 11A) Donut charts show the average relative percent of the indicated stromal populations based on FACS of dissociated benign and PCa patient tissues, n=4. The dominant population, determined by the marker of greatest intensity per cell: solid box (CD105), dashed box (CD90), double lined box (CD117), dash and dot box (Stro-1). FIG. 11B) Immunohistochemical staining of CD105 from representative core sections of tissue arrays counterstained with hematoxylin. Arrow heads indicate CD105-positive blood vessels and arrows indicate CD105-positive stromal staining, n=94. Scale bar represent 100 μm. FIG. 11C) Representative serial sections from tissue cores stained for CD105 and chromogranin A, counterstained with hematoxylin, n=39 paired tissues. See also FIG. 14. FIG. 11D) Waterfall plot shows the percent expression of chromagranin A that had co-expression of stromal CD105 on a graded scale where 0 indicates no staining and 5 indicates 100 percent staining, n=39 paired cores. FIG. 11E) Relative mRNA expression for the indicated genes is graphed for NAF and CD105-enriched CAF as mean+/−SD, n=5. Primer sequences are listed in Table 1.

FIGS. 12A-12F depict that androgen axis inhibition mediates paracrine SFRP1-mediated NED, in accordance with various embodiments of the invention. FIG. 12A) CD105 expression in human epithelial (CW22Rv1) (left column, within each group) and mouse prostatic fibroblastic cells (right column, within each group) in 3D co-culture is regulated by enzalutamide treatment, as determined by FACS analysis, n=3. FIG. 12B) Bar graph shows relative SFRP1 mRNA expression in human NAF and CAF regulated by TRC105 compared to IgG (control) treatment, n=5. FIG. 12C) Heat map shows the relative expression for the neuroendocrine gene panel in Rv1 cells, normalized to GAPDH, when treated with 0, 0.01, 0.1, 1 μg/ml SFRP1, n=5. See also FIG. 15. FIG. 12D) In a PDX model, the mice were treated with either vehicle or enzalutamide. Immunohistochemical localization of CD105 and SFRP1 in benign or PCa tissues are found in blood vessels (v), epithelia (e), and stroma (s), n=4. Scale bar represents 100 μm. FIG. 12E) Epithelial proliferation of human CW22Rv1, in 3D co-cultures with mouse prostatic fibroblasts, were co-stained for EpCam and Ki67 for FACS analysis. The cultures were treated with TRC105, M1043, and/or enzalutamide for 72 hours, n>3. See also FIG. 16. FIG. 12F) Viability of prostatic epithelia CW22Rv1, C42B, and PC3 were determined by MTT assay in the presence and absence of TRC105 and enzalutamide, n=5. Error bars are mean+/−SD and **p<0.01, ****p<0.0001, compared to control unless otherwise indicated.

FIGS. 13A-13B depict that antagonizing the androgen axis and CD105 reduced tumor growth and neuroendocrine differentiation (NED), in accordance with various embodiments of the invention. FIG. 13A) Mice were orthotopically grafted with tissue recombinants of CW22Rv1 and CAF. The mice were castrated, treated with TRC105, and/or enzalutamide. Bar graph shows tumor volumes normalized to castrated (Cx) mice. FIG. 13B) H&E staining was followed by immune-localization for phosphorylated-histoneH3 (PH-H3), TUNEL, and chromogranin A (ChromA). Scale bar represents 32 μm. The mitotic (PH-H3) and cell death (TUNEL) indexes were plotted. n>5, error bars are mean+/−SD and *p<0.05, **p<0.01, ***p<0.001, compared to control unless otherwise indicated.

FIGS. 14A-14B depict stromal CD105 expression association with neuroendocrine differentiation of the adjacent epithelia, in accordance with various embodiments of the invention. FIG. 14A) Box plot shows CD105 expression in normal and PCa tissues from the Cancer Genome Atlas Prostate Adenocarcinoma (TCGA-PRAD) data collection (n=498). FIG. 14B) Representative paired serial sections from tissue array cores stained by immunohistochemistry for CD105 or chromogranin A counterstained with hematoxylin are shown. Scale bar represents 100 μm.

FIGS. 15A-15C depict SFRP1 is associated with neuroendocrine differentiation, in accordance with various embodiments of the invention. FIG. 15A) Bar graph shows relative proliferation of Rv1 cells normalized to control and treated with the indicated concentrations of human recombinant SFRP1 or CAF conditioned media for 72 hours (mean+/−SD). FIG. 15B) Circus plot, generated using Zodiac (http://www.compgenome.org/ZODIAC), shows the relationship among related genes and the nature of the relation. Associations between copy number (CN), gene expression (GE), and methylation (Me) are denoted by lines from one node to another (p≤0.01). FIG. 15C) Bar graph shows the alteration frequency of SFRP1 mutations, deletions, and amplifications for the indicated TCGA Research Network data sets: NEPC (Trento/Cornell/Broad 2016), PCa1 (FHCRC 2016), PCa2 (MICH), PCa3 (TCGA), PCa4 (TCGA 2015), PCa5 (SU2C), PCa6 (MSKCC 2010), PCa7 (Broad/Cornell 2013), and PCa8 (Broad/Cornell 2012).

FIG. 16 depicts species specific CD105 antagonists, in accordance with various embodiments of the invention. Bar graph shows relative ID1 mRNA expression by Rv1 cells and mouse wild-type fibroblasts normalized to control. All cells were pre-treated overnight in serum free media, then incubated with BMP (50 ng/mL) in the presence or absence of differing concentrations of concentrations of TRC105 or M1043 for 6 hours (mean+/−SD). Within each group, the left column depicts human PCa and right column depicts mouse fibroblasts.

FIG. 17 depicts a schematic of epithelia following various treatments, in accordance with various embodiments of the invention.

FIGS. 18A-18F depict that radiation induced CD105 expression in prostate cancer cells support radio-resistance, in accordance with various embodiments of the invention. FIG. 18A) Cell surface CD105 expression was measured in PC3, C42b, and 22Rv1 72 hours after 4 Gy irradiation treatment by FACS analysis and compared to cells not irradiated (control). FIG. 18B) Cell surface CD105 expression was measured in cell lines following a dose range of irradiation (0, 2, 4, or 6 Gy). FIG. 18C) The durability of cell surface CD105 expression in 22Rv1 was determined 0, 0.5, 4, 8, 24, 48, 72, 120, and 168 hours following 4 Gy irradiation. CD105 cell surface expression fold change was normalized to levels expressed prior to irradiation. FIG. 18D) Western blot for phosphorylated-Smad1/5 was measured in CW22Rv1 cells in the presence or absence of serum starvation and treatment with 50 ng/ml BMP4 or 1 μg/ml TRC105. β-actin expression served as the loading control. FIG. 18E) Annexin-V expression was measured in 22Rv1 cells by FACS analysis 5 days following 4 Gy irradiation in the presence and absence of TRC105. FIG. 18F) Clonogenic assay was measured 10 days following irradiation of CW22Rv1 and C42b cells in a dose range of 0 to 6 Gy in the presence of 1 μg/ml IgG or TRC105. Data are reported as a mean +/−S.D. (**p<0.01, ***p<0.001).

FIG. 19 depicts in accordance with various embodiments of the invention, ID1 mRNA expression measured in CW22Rv1 under serum free conditions with 50 ng/ml BMP4 under serum-free conditions. IgG in the context of increasing doses of TRC105 (0.05, 0.1, 0.5, 1, 5, or 10 μg/ml). ID1 mRNA expression was normalized to GAPDH. (**p<0.01, ****p<0.0001).

FIGS. 20A-20F depict that radiation induces BMP-mediated SIRT1 expression, in accordance with various embodiments of the invention. FIG. 20A) Western blot for SIRT1 expression measured in 22Rv1 cells following serum starvation and treatment with 50 ng/ml BMP4 for 4 hours. Phosphorylated Smad1/5 and β-actin was measured concurrently. FIG. 20B) SIRT1 mRNA expression was measured in CW22Rv1 under serum free conditions with 50 ng/ml BMP4, IgG in the context of increasing doses of TRC105 (0.05, 0.1, 0.5, 1, 5, or 10 μg/ml). SIRT1 mRNA expression was normalized to GAPDH and to serum treated control. FIG. 20C) Fold Change of SIRT1 mRNA in benign prostate and prostate cancer patients, obtained from R2-Genomics analysis is expressed (n=95). FIG. 20D) Immunohistochemical localization for SIRT1 expression in benign and prostate cancer tissues is indicated by arrows (Human Protein Atlas). The “e” and “s” indicate epithelia and stromal compartments in the tissues, respectively. FIG. 20E) SIRT1 mRNA expression was measured 72 hours following irradiation of 22Rv1 in a dose range of 0-6 Gy. FIG. 20F) SIRT1 mRNA expression was measured in a time course 0-72 hours following 4 Gy irradiation. SIRT1 mRNA expression was normalized to GAPDH and to untreated, 0 Gy. Data are reported as a mean+/−S.D. of 3 independent experiments (***p<0.001, ****p<0.0001).

FIGS. 21A-21C depict SIRT1 mRNA expression was quantitated, in accordance with various embodiments of the invention. FIG. 21A) C4-2B were irradiated (0, 2, 4, or 6 Gy) and SIRT1 expression measured 72 hours post irradiation. FIG. 21B) C4-2B cells were irradiated (4 Gy) and SIRT1 expression measured 0, 0.5, 4, 8, 24, 48, and 72 hours post-radiation. FIG. 21C) 22Rv1 were pre-treated with 1 μg/ml IgG or TRC105 24 hours prior to irradiation with 4 Gy and compared for relative SIRT1 mRNA expression 72 hours after irradiation. SIRT1 mRNA was normalized to GAPDH and to 0 Gy control.

FIGS. 22A-22C depict that CD105 induces transient DNA damage and cell cycle arrest, in accordance with various embodiments of the invention. 22Rv1 were pre-treated with 1 μg/ml TRC105 24 hours prior to irradiation with 4 Gy. FIG. 22A) γ-H2AX or p53 bp were imunolocalized at 4, 24, and 48 hours post radiation. Foci per nuclei were quantified (n=100). FIG. 22B) Comet assay was performed 30 minutes and 24 hours following irradiation. The tail moment was quantified (n=50). FIG. 22C) Cell cycle analysis was performed on 22Rv1 at 0, 4, 8, and 24 hours post radiation in the presence of IgG or TRC105 (n=3) in 3 independent experiments. (***p<0.001, ****p<0.0001).

FIGS. 23A-23E depict clonogenic survival assays, in accordance with various embodiments of the invention. Assays were performed on cell lines with p53 null prostate cancer cell line, FIG. 23A) PC3 and two p53 mutant pancreatic cancer cell lines, FIG. 23B) MIAPACA-2 and FIG. 23C) HPAF-II with indicated doses of radiation. Breast cancer cell lines with intact p53, FIG. 23D) MCF7 and mutant yet functional p53, FIG. 23E) MDA-MB23 were however radio-sensitized by the 1 μg/ml TRC105.

FIGS. 24A-24D depict that PGC1α and mitochondrial biogenesis are regulated by BMP/CD105. 22Rv1 cells were incubated with IgG or TRC105 with or without 4 Gy irradiation. All measurements were made 72 hours post radiation. FIG. 24A) Western blot for whole cell lysate, nuclear and cytoplasmic fractions were independently analyzed for PGC1α expression. Loading controls included β-actin (whole cell), lamin B (nuclear marker), and Rho A (cytoplasm marker). FIG. 24B) Immunofluorescent localization of PGC1α was visualized with DAPI nuclear counterstain. FIG. 24C) mRNA expression of PGC1α target genes, NRF1, MTFA, and CPT1C was measured. mRNA expression was normalized to GAPDH and untreated IgG 0 Gy. FIG. 24D) Mitochondrial DNA (mtDNA) was measured from total DNA extracts and normalized to nuclear DNA and compared to untreated IgG 0 Gy. Data are reported as means+/−S.D. of 3 independent experiments. (***p<0.001, ****p <0.0001).

FIGS. 25A-25B depict 22Rv1 were treated with 1 μg/ml IgG or TRC105 prior to irradiation with 4Gy, in accordance with various embodiments of the invention. Lysate was collected 72 hours post irradiation for Western blot. FIG. 25A) Blots were probed for a cocktail of mitochondrial complex proteins. Protein levels of MTCO1 of complex-IV and NDUFB8 of complex-I were normalized to ponceau. FIG. 25B) MTCO1 and NDUFB8 were significantly lower in 4Gy+ TRC105 compared to radiation alone. Within each group, the first/left column depicts 0Gy+IgG, the second column depicts 0Gy+TRC105, the third column depicts 4Gy+IgG and the last/right column depicts 4Gy+TRC105. (**p<0.01, ***p<0.001)

FIGS. 26A-26D depict metabolic changes induced by CD105 antagonism, in accordance with various embodiments of the invention. Cells were analyzed for mito-stress test by Seahorse-XF 168 hours following 4 Gy of radiation in the presence of IgG or TRC105. FIG. 26A) Basal respiration, non-mito respiration, proton leak, spare respiratory capacity, FIG. 26B) extracellular acidification rate (ECAR) and FIG. 26C) mitochondrial dependent ATP production were quantitated using Wave 2.3.0 analysis. Data are reported as mean+/−S.D. of a representative experiment (n=5) of 3 independent experiments. FIGS. 26A-26C, the first/left column depicts 0Gy+IgG, the second column depicts 0Gy+TRC105, the third column depicts 4Gy+IgG and the last/right column depicts 4Gy+TRC105. FIG. 26D) 22Rv1 cells treated with IgG, TRC105 or nicotinamide were irradiated (4 Gy). Total cellular ATP was measured 0, 24, 72, 120, and 168 hours post radiation. Within each group, the left column is IgG, middle column is TRC105 and the right column is nicotinamide. Data are reported as mean+/−S.D. of 3 independent experiments. (***p<0.001, ****p<0.0001).

FIG. 27 depicts the role of ATP depletion on radiation sensitivity, in accordance with various embodiments of the invention. 22Rv1 cells were treated with indicated doses of ATPase inhibitor, oligomycin and exposed to 4Gy irradiation. Within each group, the left column is 0 Gy and the right column is 4 Gy. Cell counts were performed 72 hrs. following irradiation. (**p<0.01, ***p<0.001)

FIGS. 28A-28B depict that antagonizing CD105 confers radio-sensitivity in vivo, in accordance with various embodiments of the invention. Tumor volumes were longitudinally measured. When tumor average volume reached 80 mm mice were treated with IgG or TRC105 in the context of radiation (2 Gy for 5 days). Tumors were harvested 15 days after the first dose of radiation. FIG. 28A) Tumor volume fold change was normalized to the first dose of radiation (day 1, *** p<0.001). FIG. 28B) Each treatment was compared for doubling of tumor volume as a function of time as depicted in the cumulative incidence plot.

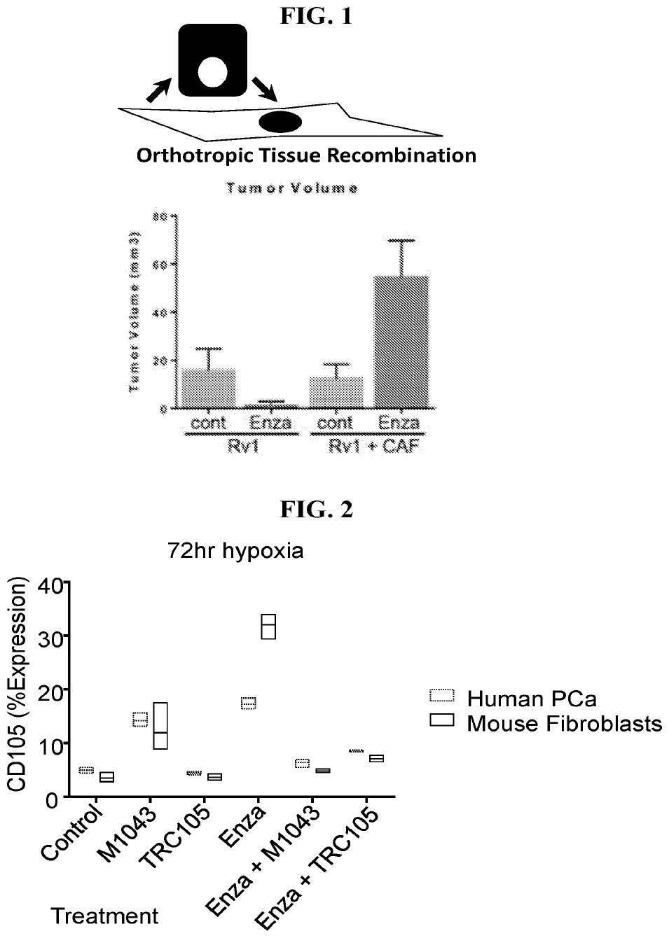

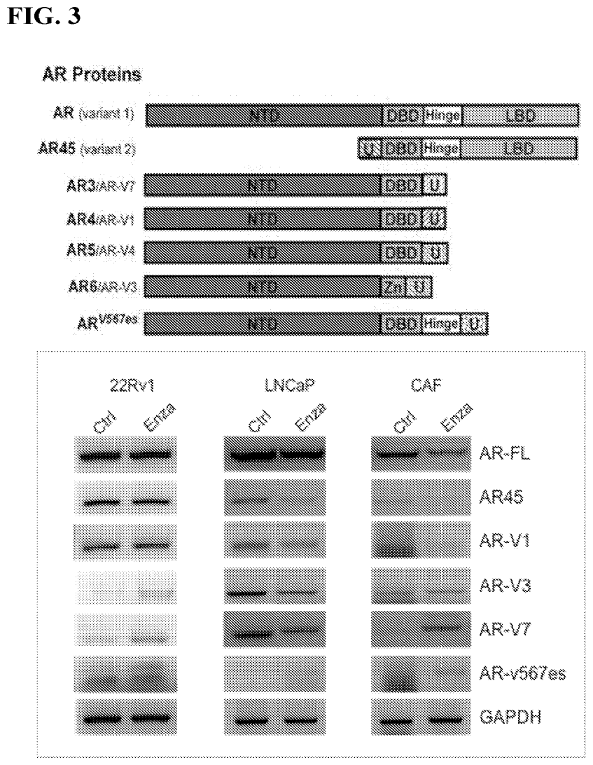

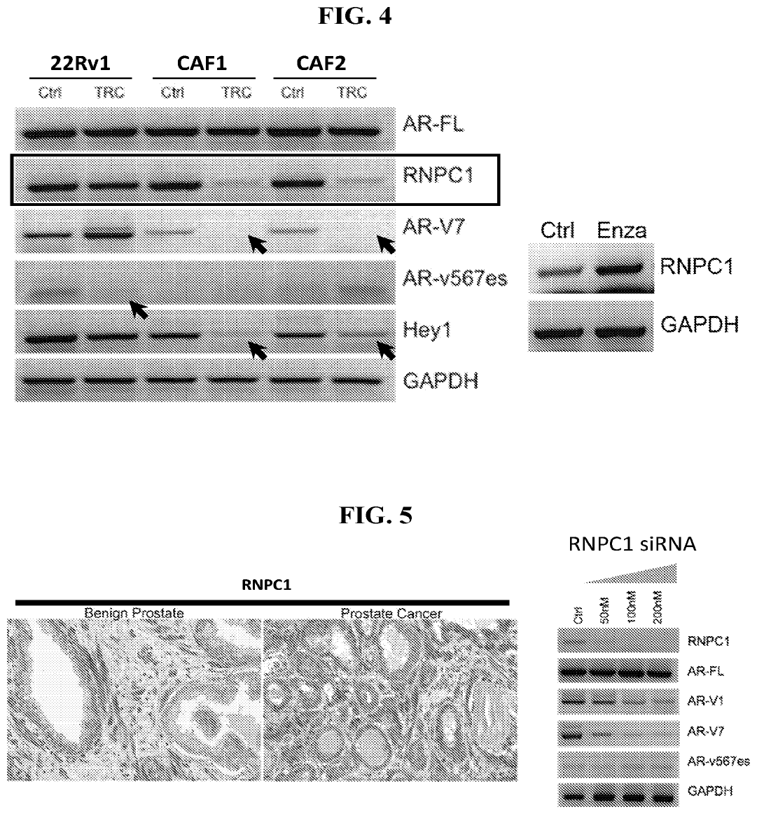

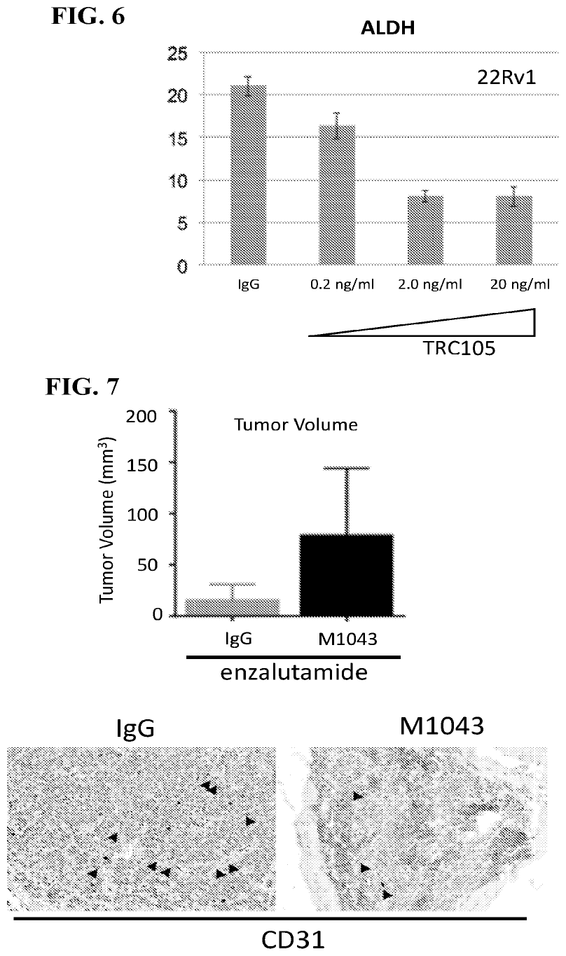

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Kohler and Milstein, Derivation of specific antibody producing tissue culture and tumor lines by cell fusion, Eur. J. Immunol. 1976 Jul., 6(7):511-9; Queen and Selick, Humanized immunoglobulins, U.S. Pat. No. 5,585,089 (1996 Dec); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“PCa” as used herein refers to prostate cancer.

“ATT” as used herein refers to androgen targeted therapy.

“CAF” as used herein refers to carcinoma associated fibroblasts.

“CRPC” as used herein refers to castration resistant prostate cancer.

“NED” as used herein refers to neuroendocrine differentiation.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of cancer, delay or slowing of cancer, and amelioration or palliation of symptoms associated with cancer.

“Diseases”, “conditions” and “disease conditions,” as used herein may include, but are in no way limited to any form of malignant neoplastic cell proliferative disorders or diseases. Examples of such disorders include but are not limited to cancer and tumor.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. A subject that has a cancer or a tumor is a subject having objectively measurable cancer cells present in the subject's body. Included in this definition are benign and malignant tumors, as well as dormant tumors or micrometastasis. Cancers which migrate from their original location and seed vital organs can eventually lead to the death of the subject through the functional deterioration of the affected organs. As used herein, the term “invasive” refers to the ability to infiltrate and destroy surrounding tissue. Melanoma is an invasive form of skin tumor. As used herein, the term “carcinoma” refers to a cancer arising from epithelial cells. Examples of cancer include, but are not limited to, breast cancer, bladder cancer, lung cancer, colorectal cancer, colon cancer, rectal cancer, pancreatic cancer, liver cancer, renal cancer, renal cell carcinoma, carcinoma, melanoma, sarcoma, head and neck, glioblastoma, and prostate cancer, including but not limited to androgen-dependent prostate cancer and androgen-independent prostate cancer. As used herein, the term “administering,” refers to the placement of an agent or a composition as disclosed herein into a subject by a method or route which results in at least partial localization of the agents or composition at a desired site.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., cancer) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. For example, a subject can be one who exhibits one or more symptoms for a condition or one or more complications related to the condition or a subject who does not exhibit symptoms. A “subject in need” of diagnosis or treatment for a particular condition can be a subject suspected of having that condition, diagnosed as having that condition, already treated or being treated for that condition, not treated for that condition, or at risk of developing that condition.

The term “functional” when used in conjunction with “equivalent”, “analog”, “derivative” or “variant” or “fragment” refers to an entity or molecule which possess a biological activity that is substantially similar to a biological activity of the entity or molecule of which it is an equivalent, analog, derivative, variant or fragment thereof.

In accordance with the present invention, the term “radiation therapy” or “radiotherapy” refers to a cancer treatment that uses high-energy particles or waves, such as x-rays, gamma rays, electron beams, or protons, to destroy or damage cancer cells or prevent them from growing and dividing. Other names for radiation therapy include irradiation or x-ray therapy. Radiation can be given alone or used with other treatments, such as surgery or chemotherapy. Depending on the cancer type and location, there are also three different ways to give radiation therapy: external radiation, internal radiation, and systemic radiation. Sometimes a patient gets more than one type of radiation therapy for the same cancer.

External radiation (or external beam radiation) therapy uses a machine that directs high-energy rays from outside the body into the tumor. External radiation therapy is usually given with a machine called a linear accelerator (often called a “linac” for short). Types of external radiation therapy include but are not limited to standard external beam radiation therapy, conventional external beam radiation therapy (2DXRT), image guided radiotherapy (IGRT), three-dimensional conformal radiation therapy (3D-CRT), intensity modulated radiation therapy (IMRT), helical tomotherapy, volumetric modulated arc therapy (VMAT), particle therapy, proton beam therapy, carbon ion therapy, conformal proton beam radiation therapy, auger therapy (AT), intraoperative radiation therapy (IORT), stereotactic radiation therapy, stereotactic radiosurgery (SRS), and stereotactic body radiation therapy (SBRT). There are three different ways of giving SRS: the most common type uses a movable linac that's controlled by a computer to move around to target the tumor from many different angles (e.g., X-KNIFE, CYBERKNIFE, and CLINAC); the second type is the GAMMA KNIFE, which uses about 200 small beams aimed at the tumor from different angles for a short period of time to deliver a large dose of radiation; and the third type uses heavy charged particle beams (like protons or helium ion beams) to deliver radiation to the tumor.

Internal radiation therapy (also called brachytherapy) uses a radioactive source that's put inside the body in or near the tumor. The main types of brachytherapy are intracavitary radiation and interstitial radiation. Both of these methods use radioactive implants such as pellets, seeds, ribbons, wires, needles, capsules, balloons, or tubes. High-dose-rate (HDR) brachytherapy allows a person to be treated for only a few minutes at a time with a powerful radioactive source that's put in the applicator, and the source is removed after several minutes. Low-dose-rate brachytherapy uses the implant to give off lower doses of radiation over a longer period of time.

Systemic radiation therapy uses radioactive drugs (called radiopharmaceuticals) to treat certain types of cancer. These drugs can be given by mouth or put into a vein; they then travel throughout the body. These radiation sources are in the form of a liquid made up of a radioactive substance, and they are sometimes attached with a targeting agent that guides them to cancers and tumors. For example, a monoclonal antibody can be used to target the radioactive substance to the cancer cells, that is, a radioimmunotherapy.

Radioimmunotherapy is a type of systemic radiation therapy, in which monoclonal antibodies are attached to the radioactive substance. Monoclonal antibodies are laboratory-made proteins designed to recognize specific factors only found in cancer cells, and they can deliver low doses of radiation directly to the tumor while leaving noncancerous cells alone. Exemplar radioimmunotherapy include ibritumomab (ZEVALIN) and tositumomab (BEXXAR). Radioisotope therapies (e.g., radioactive iodine, strontium, samarium, strontium-89, samarium (¹⁵³Sm) lexidronam, and radium) are another type of systemic radiation used to treat certain types of cancers, such as thyroid, bone, and prostate cancers. Examples of radioisotope therapies include but are not limited to metaiodobenzylguanidine (MIBG), iodine-131, hormone-bound lutetium-177 and yttrium-90, yttrium-90 radioactive glass or resin microspheres, ibritumomab tiuxetan (Zevalin, an anti-CD20 monoclonal antibody conjugated to yttrium-90), tositumomab/iodine (1311) tositumomab regimen (BEXXAR, a combination of an iodine-131 labeled and an unlabeled anti-CD20 monoclonal antibody)

Radiation therapy dosages may be given in different ways, such as hyperfractionated radiotherapy and hypofractionated radiotherapy. In hyperfractionated radiotherapy, the total dose of radiation is divided into small doses and treatments are given more than once a day. Hyperfractionated radiation therapy is given over the same period of time (days or weeks) as standard radiation therapy. It is also called superfractionated radiation therapy. One type of hyperfractionated radiotherapy is continuous hyperfractionated accelerated radiotherapy (CHART). CHART without treatments at the weekends is called CHARTWEL. In hypofractionated radiotherapy, the total dose of radiation is divided into large doses and treatments are given once a day or less often. Hypofractionated radiation therapy is given over a shorter period of time (fewer days or weeks) than standard radiation therapy.

In various embodiments, the inventors antagonize endoglin (e.g., using TRC105) to support radiation sensitivity. There are a number of novel aspects to our findings regarding the role of BMP signaling in radiation therapy of solid tumors: 1) we have found for the first time that BMP signaling is up regulated as a result of radiation; 2) BMP signaling can also support radiation survival; 3) further, BMP signaling by the carcinoma associated fibroblastic cells is a mediator of tumor survival; and 4) antagonizing BMP signaling by antagonizing endoglin results in tumor sensitization to radiation as a result of interactions of the tumor with its microenvironment. These findings can be applicable to any solid tumor type including colon, breast, melanoma, and lung.

In various embodiments, we antagonize endoglin (e.g., using TRC105) to limit the expression of androgen receptor splice variants responsible for the resistance to hormonal therapy. Androgen deprivation therapy (ADT), include enzalutamide and abiraterone, is the most common treatment for recurrent prostate cancer following primary ablation therapy. ADT is associated with the gain of improperly spliced AR expression. TGF-β stromal responsiveness is shown to determine androgen sensitivity in the adjacent prostatic epithelia. The loss of TGF-β responsiveness in the prostate cancer stromal tissues is associated with the expression of androgen receptor splice variant (ARv). The ARv can translocate to the nucleus and activate androgen responsive genes in a ligand independent manner—thus eliciting therapeutic resistance. In the past, IL-6 expression by prostate cancer epithelia has been shown to result in ARv expression in the epithelia itself in contributing to ADT resistance. We have found that the loss of TGF-β responsiveness in the prostatic fibroblasts result in coincident Notch and CD105 signaling in the mechanism of ARv expression. We found that antagonizing endoglin (e.g., using TRC105) can down regulate Notch and IL-6 mediated ARv expression. Our in vivo data demonstrated that the combination of TRC105 with ADT is superior to either one alone in prostate cancer models. Similar results can be had with breast cancer in the context of SERMs (selective estrogen receptor modulator) for ER+ cancers.

In various embodiments, we antagonize endoglin (e.g., using TRC105) to reduce stem properties of cancer epithelia. Our data show that cancer stem cell markers (e.g., CD44, ALDH, Oct4, and Sox) as well as sphere-forming units (another measure of stem features) are down regulated in prostatic epithelia. The significance of this observation is that the gain of stem features in cancer cells is associated with therapeutic resistance and metastatic progression. Thus, to treat cancers, we combine TRC105 with chemotherapy (e.g., taxanes, vinblastine, and platinum based drugs).

In various embodiments, we antagonize endoglin (e.g., using TRC105) to limit the development of local recurrence in breast cancer patients who undergo mammoplasty surgery (radical or lobe) to remove the tumor. Proliferating vasculature (often expressing CD105) is demonstrated to promote the proliferation of adjacent breast cancer cells. Thus, inhibiting such vascular endothelia with TRC105 can be beneficial. As with others, other solid tumors may similarly benefit from prophylactic use of TRC105 following surgical resection.

Prostate cancer (PCa) is a heterogeneous disease that results in the second highest cancer mortality in men. The standard of care for most localized prostate cancer is radiotherapy or surgical resection. Radiation is also used as an adjuvant therapy to surgery and even in a palliative setting for bone metastasis. Up to 30% of localized prostate cancer patients treated with radiation ablative therapy develop recurrent radio-resistant disease. Further, 50% of patients that undergo salvage radiation therapy after biochemical recurrence will have disease progression. Radio-toxicity is a significant obstacle in achieving curative doses.

The standard of care for recurrent PCa is the disruption of androgen signaling. Therapeutics for late stage PCa target the androgen axis by blocking androgen synthesis or the androgen receptor. Despite the initial efficacy of ATT, PCa becomes resistant, and many patients develop castration resistant prostate cancer (CRPC) with characteristic neuroendocrine features. The eventual development of resistance to androgen targeted therapy (ATT) has no curative approaches currently, and thus there is an unmet need in the art.

The inventors identified different fibroblastic populations that make up what we term CAF, based on its ability to support tumor expansion. Of the different fibroblastic populations, identified through common mesenchymal cell surface markers, those expressing CD105 were found to be critical for the expansion of existing tumor epithelia and further promote neuroendocrine features in PCa in four ways: 1) the recombination of two non-tumor potentiating NAF and CAF^{HiP} with PCa epithelia yielded tumors similar to tumor inductive CAF, 2) enrichment of CD105 identified in human PCa tissues is further enhanced by ATT, 3) localization of CD105⁺ CAF circumscribe areas of NED, and 4) use of CD105 neutralizing antibody in 3D cultures and mouse experiments reduced epithelial expansion in the context of androgen-axis targeting. The inventors correlated the reduced CD105 population in the CAF^{HiP} with reduced in vivo tumor expansion. The cell population drift associated with culturing was exploited here as it revealed changes in CD105. However, this culture-associated drift included changes in the CD90⁺ population, contrary to that observed in tissues. Stromal CD105 changes induced by ATT were found to mediate epithelial NED through paracrine signaling.

Without being bound to any particular theory, the combining of ATT and CD105 antagonism is an example of synthetic lethality. ATT resistance in advanced CRPC is known to arise due to variable responses in the context of tumor heterogeneity. The inventors found elevated CD105 to be a mediator of ATT-induced NED. In these studies, the inventors identified that the CD105 fibroblastic population expresses SFRP1, as a potential means of surviving in androgen deprived conditions. Antagonizing CD105 inhibited SFRP1 expression and NED of the prostate tumors. Without being bound to any particular theory, it is likely that SFRP1 is involved in balancing the maintenance of proliferation versus stem-like features. In previous studies, the inventors found that SFRP1 potentiates a neuroendocrine signature in PCa cells inclusive of classic markers aurora kinase, n-myc, and secretogranin-3 (Beltran et al., 2012, J Amer Soc Clin Oncology 30, e386-389). Furthermore, while in the tissue recombination xenograft model of CRPC, where castration followed by enzalutamide treatment did not significantly decrease tumor growth, the same epithelia in monolayer devoid of stroma was sensitive to enzalutamide treatment. Thus, without being bound to any particular theory, the role of the stromal fibroblasts is necessary in paracrine-mediated development of CRPC.

Endoglin (CD105), a type III TGFβ/BMP co-receptor, originally identified in proliferating endothelia, is up-regulated in several cancers including prostate cancer. CD105 antagonizes TGF-β signaling and promotes bone morphogenic protein (BMP) signaling and antagonizing TGF-β signaling. CD105 expression on various cancers has correlated with progression, metastasis, aggressiveness, and evasion to conventional therapeutics. Without being bound to any particular theory, the inventors believe that targeting CD105 sensitizes prostate cancer to cancer therapies. To demonstrate, the inventors used a partially humanized monoclonal antibody that blocks BMP signaling, TRC105.

As described herein, the inventors identified that CD105-expressing prostatic fibroblasts are enriched in tumor inductive CAF, further amplified by androgen targeted therapy (ATT), and contribute to CRPC in a paracrine manner. Fibroblastic CD105 enhances prostatic tumor progression and neuroendocrine differentiation. Antagonizing CD105 with a neutralizing antibody down-regulated SFRP1 expression by CAF.

Furthermore, the inventors demonstrate that blocking BMP/CD105 signaling using TRC105, inhibits SIRT1 expression and its downstream regulated proteins, p53 and peroxisome proliferator-activated receptor gamma co-activator 1-alpha (PGC1α).

Thus, antagonizing CD105 sensitized PCa tumors to ATT and radiation.

The present invention is based, at least in part, on these findings. Embodiments address the need in the art for method of sensitizing a cancer in a subject and methods of treating, slowing the progression of, reducing the severity of, preventing the recurrence of, and/or reducing the recurrence likelihood of a cancer in a subject. Embodiments further provide for a method of preventing the recurrence of and/or reducing the recurrence likelihood of a cancer in a subject who has been treated with a cancer therapy.

### Method of Sensitizing a Cancer

Various embodiments of the present invention provide for a method of sensitizing a cancer in a subject in need thereof, comprising: providing a CD105 antagonist; and administering the CD105 antagonist to the subject, thereby sensitizing the cancer. In various embodiments, the method further comprises administering a cancer therapy. In various embodiments, the method further comprises identifying a subject in need of sensitizing a cancer to cancer treatment before administering the CD105 antagonist.

Various embodiments of the present invention provide for a method of sensitizing a cancer in a subject in need thereof, comprising: administering the CD105 antagonist to the subject, thereby sensitizing the cancer. In various embodiments, the method further comprises administering a cancer therapy. In various embodiments, the method further comprises identifying a subject in need of sensitizing a cancer to cancer treatment before administering the CD105 antagonist.

Various embodiments of the present invention provide for a method of sensitizing a cancer in a subject who is not responsive to a cancer therapy, comprising: administering the CD105 antagonist to the subject, thereby sensitizing the cancer. In various embodiments, the method further comprises administering a cancer therapy.

Various embodiments of the present invention provide for a method of sensitizing a cancer in a subject in need thereof, comprising: identifying a subject in need of sensitizing a cancer to cancer treatment before administering the CD105 antagonist; and administering the CD105 antagonist to the subject, thereby sensitizing the cancer. In various embodiments, the method further comprises administering a cancer therapy. In various embodiments, the subject has previously received cancer therapy.

In various embodiments, the cancer is prostate cancer, breast cancer, bladder cancer, lung cancer, colorectal cancer, pancreatic cancer, liver cancer, renal cancer, renal cell carcinoma, melanoma, sarcoma, head and neck cancer, glioblastoma, or a combination thereof. In various embodiments, the cancer is resistant to radiation and/or androgen targeted therapy. In various embodiments, the cancer is prostate cancer. In various embodiments, the cancer is castrate resistant prostate cancer (CRPC).

In various embodiments, the CD105 antagonist is an antibody specifically binding to CD105 or an antigen-binding fragment thereof. In various other embodiments, the CD105 antagonist is TRC105 or an antigen-binding fragment thereof.

In various embodiments, the cancer therapy is radiotherapy, chemotherapy, hormone therapy, or surgery, or a combination thereof. In various embodiments, the subject is treated by the administration of the CD105 antagonist and the cancer therapy.

In various embodiments, the present invention provides a method of sensitizing a cancer in a subject to a cancer therapy. The method comprises: providing a CD105 antagonist; and administering the CD105 antagonist to the subject, thereby sensitizing the cancer to the cancer therapy. In various embodiments, the cancer therapy is radiotherapy, chemotherapy, hormone therapy, or surgery, or a combination thereof. In various embodiments, the method further comprises treating the subject with the cancer therapy.

### Methods of Treating

Various embodiments of the present invention provide for a method of treating, slowing the progression of, reducing the severity of, preventing the recurrence of, and/or reducing the recurrence likelihood of a cancer in a subject in need thereof, comprising: administering a CD105 antagonist to the subject; and administering a cancer therapy to the subject, thereby treating, slowing the progression of, reducing the severity of, preventing the recurrence of, and/or reducing the recurrence likelihood of the cancer in the subject.

In various embodiments, the cancer is prostate cancer, breast cancer, bladder cancer, lung cancer, colorectal cancer, pancreatic cancer, liver cancer, renal cancer, renal cell carcinoma, melanoma, sarcoma, head and neck cancer, glioblastoma, or a combination thereof. In various embodiments, the cancer is resistant to radiation and/or androgen targeted therapy. In various other embodiments, the cancer is prostate cancer. In various embodiments, the cancer is castrate resistant prostate cancer (CRPC).

In various embodiments, the CD105 antagonist is an antibody specifically binding to CD105 or an antigen-binding fragment thereof. In various other embodiments, the CD105 antagonist is TRC105 or an antigen-binding fragment thereof.

In various embodiments, the cancer therapy is radiotherapy, chemotherapy, hormone therapy, or surgery, or a combination thereof.

In various embodiments, the present invention provides a method of treating, slowing the progression of, reducing the severity of, preventing the recurrence of, and/or reducing the recurrence likelihood of a cancer in a subject. The method comprises: providing a CD105 antagonist; administering the CD105 antagonist to the subject, thereby sensitizing the cancer to a cancer therapy; and administering the cancer therapy to the subject, thereby treating, slowing the progression of, reducing the severity of, preventing the recurrence of, and/or reducing the recurrence likelihood of the cancer in the subject. In various embodiments, the cancer therapy is radiotherapy, chemotherapy, hormone therapy, or surgery, or a combination thereof.

In various embodiments, the present invention provides a method of treating, slowing the progression of, reducing the severity of, preventing the recurrence of, and/or reducing the recurrence likelihood of a cancer in a subject. The method comprises: providing a CD105 antagonist; administering a CD105 antagonist to the subject; and administering a cancer therapy to the subject, thereby treating, slowing the progression of, reducing the severity of, preventing the recurrence of, and/or reducing the recurrence likelihood of the cancer in the subject. In various embodiments, the cancer therapy is radiotherapy, chemotherapy, hormone therapy, or surgery, or a combination thereof.

In various embodiments, the present invention provides a method of treating, slowing the progression of, reducing the severity of, preventing the recurrence of, and/or reducing the recurrence likelihood of castrate resistant prostate cancer in a subject. The method comprises: administering a CD105 antagonist to the subject; and administering an androgen targeted therapy to the subject, thereby treating, slowing the progression of, reducing the severity of, preventing the recurrence of, and/or reducing the recurrence likelihood of castrate resistant prostate cancer in the subject. In various embodiments, the androgen targeted therapy is enzalutamide. In various embodiments, the CD105 antagonist is TRC105 or an antigen-binding fragment thereof. In various embodiments, the antigen is CD105. In various embodiments, the antigen is endoglin.

Preventing and/or Reducing Likelihood of Recurrence

Various embodiments of the present invention provide for a method of preventing the recurrence of and/or reducing the recurrence likelihood of a cancer in a subject who has been treated with a cancer therapy, comprising: administering the CD105 antagonist to the subject; and administering a cancer therapy, thereby preventing the recurrence of and/or reducing the recurrence likelihood of the cancer.

In various embodiments, the cancer is prostate cancer, breast cancer, bladder cancer, lung cancer, colorectal cancer, pancreatic cancer, liver cancer, renal cancer, renal cell carcinoma, melanoma, sarcoma, head and neck cancer, glioblastoma, or a combination thereof. In various embodiments, the cancer is resistant to radiation and/or androgen targeted therapy. In various embodiments, the cancer is prostate cancer. In various embodiments the cancer is castration resistant prostate cancer.

In various embodiments, the CD105 antagonist is an antibody specifically binding to CD105 or an antigen-binding fragment thereof. In various embodiments, the CD105 antagonist is TRC105 or an antigen-binding fragment thereof. In various embodiments, the antigen is CD105. In various embodiments, the antigen is endoglin.

In various embodiments, the cancer therapy is radiotherapy, chemotherapy, hormone therapy, or surgery, or a combination thereof. In various embodiments, the cancer therapy is the same as a cancer therapy previously administered to the subject. In various embodiments, the cancer therapy is different from a cancer therapy previously administered to the subject.

In various embodiments, the present invention provides a method of preventing the recurrence of and/or reducing the recurrence likelihood of a cancer in a subject. The method comprises: providing a CD105 antagonist; administering the CD105 antagonist to the subject, thereby preventing the recurrence of and/or reducing the recurrence likelihood the cancer. In various embodiments, the subject has been treated with a cancer therapy. In various embodiments, the cancer therapy is radiotherapy, chemotherapy, hormone therapy, or surgery, or a combination thereof. In some embodiments, the cancer therapy is a surgery that removes the cancer or at least a portion of the cancer. In some embodiments, the subject has been treated with a surgery that removes the cancer or a surgery that removes at least a portion of the cancer. In one embodiment, the surgery is mastectomy. In another embodiment, the surgery is orchiectomy.

Various embodiments of the present invention provide for a method of preventing the recurrence of and/or reducing the recurrence likelihood of castration resistant prostate cancer in a subject who has been treated with a cancer therapy, comprising: administering the CD105 antagonist to the subject; and administering a cancer therapy, thereby preventing the recurrence of and/or reducing the recurrence likelihood of the castration resistant prostate cancer.

In various embodiments, the subject is a human. In various embodiments, the subject is a mammalian subject including but not limited to human, monkey, ape, dog, cat, cow, horse, goat, pig, rabbit, mouse and rat.

In various embodiments, the cancer is prostate cancer, breast cancer, bladder cancer, lung cancer, colorectal cancer, pancreatic cancer, liver cancer, renal cancer, renal cell carcinoma, melanoma, sarcoma, head and neck cancer, glioblastoma, or a combination thereof. In some embodiments, the cancer is prostate cancer. In various embodiments the cancer is castration resistant prostate cancer. In other embodiments, the cancer is breast cancer. In various embodiments, the CD105 antagonist and the cancer therapy are administered sequentially, alternatively, or concurrently. In some embodiments, the CD105 antagonist and the cancer therapy are administered sequentially. In some embodiments, the CD105 antagonist and the cancer therapy are administered alternatively. In some embodiments, the CD105 antagonist and the cancer therapy are administered concurrently. In various embodiments, more than one cancer therapy can be administered.

The term “sequentially” or “sequentially administered” as used herein refers to the administration of a therapeutic agent (i.e., CD105 antagonist or a cancer therapy) in order, such that a first therapeutic agent is administered followed by a second therapeutic agent. For example, the CD105 antagonist is administered followed by the administration of the cancer therapy or vice versa. In various embodiments, the administration of the first therapeutic agent can be administered immediately, 1 minute, 5 minutes, 10 minutes, 20 minutes, 30 minutes or 45 minutes before the administration of the second therapeutic agent. In other embodiments, the first therapeutic agent is administered 1 hour, 2 hours, 3 hours, 4 hours, 5 hours, 6 hours, 12 hours or 24 hours before the second therapeutic agent. In still other embodiments, the first therapeutic agent is administered 2 days, 3 days or 4 days before the second therapeutic agent.

The term “alternatively” as used herein refers to the administration of the first therapeutic agent over the second therapeutic agent, or vice versa.

The term “concurrently” as used herein refers to the administration of the first therapeutic agent and the second therapeutic agent at the same time/simultaneously. In some embodiments, the therapeutic agents are in a single composition. In various embodiments, the therapeutic agents are in separate compositions.

In various embodiments, the CD105 antagonist is administered once a day, twice a day, once a week, twice a week, once every two weeks, once every 3 weeks, or once a month. In various embodiments, the CD105 antagonist is administered once per week. In various other embodiments, the CD105 antagonist is administered once every two weeks. In various embodiments, the CD105 antagonist is administered for a period of time until the tumor is no longer detectable. In some embodiments, the detection of the tumor includes, but is not limited to radiography and/or blood tests.

In various embodiments, the cancer therapy is administered for a duration that is established for the standard of care for the particular therapy. In various embodiments, the cancer therapy is administered for 1 month, 2 months, 3 months, 4 months, 5 month, 6 months, 7 months, 8 months, 9 months, 10 months, 11, months, 12 months or combinations thereof. In various embodiments, the cancer therapy is administered for 2 years, 3 year, 4 years, 5 years, 6 years, 7 years, 8 years, 9 years, 10 years or combinations thereof.

In various embodiments, the CD105 antagonist is administered in parallel with the cancer therapy. For example, if the CD105 antagonist is administered once a week and the cancer therapy is administered for a month, then the CD105 antagonist is administered four times to the subject in need thereof.

In various embodiments, the CD105 antagonist is administered once per week and the cancer therapy is administered for one month. In various embodiments, the CD105 antagonist is administered once per week and the cancer therapy is administered for two months. In various embodiments, the CD105 antagonist is administered once per week and the cancer therapy is administered for four months. In various embodiments, the CD105 antagonist is administered once per week and the cancer therapy is administered for eight months. In various embodiments, the CD105 antagonist is administered once per week and the cancer therapy is administered for one year. In various embodiments, the CD105 antagonist is administered once per week and the cancer therapy is administered for more than one year.

In various other embodiments, the CD105 antagonist is administered once every two weeks and the cancer therapy is administered for one month. In various embodiments, the CD105 antagonist is administered once every two weeks and the cancer therapy is administered for two months. In various embodiments, the CD105 antagonist is administered once every two weeks and the cancer therapy is administered for four months. In various embodiments, the CD105 antagonist is administered once every two weeks and the cancer therapy is administered for eight months. In various embodiments, the CD105 antagonist is administered once every two weeks and the cancer therapy is administered for one year. In various embodiments, the CD105 antagonist is administered once every two weeks and the cancer therapy is administered for more than one year.

In various embodiments, the CD105 antagonist is administered before, during, or after administering the cancer therapy. In some embodiments, the CD105 antagonist is administered before administering the cancer therapy. In some embodiments, the CD105 antagonist is administered during administering the cancer therapy. In some embodiments, the CD105 antagonist is administered after administering the cancer therapy.

In various embodiments, the CD105 antagonist is an antibody specifically binding to CD105 or an antigen-binding fragment thereof. In some embodiments, the antibody is a polyclonal antibody. In other embodiments, the antibody is a monoclonal antibody. In various embodiments, the antibody can be of any animal origin. Examples of the animal origin include but are not limited to human, non-human primate, monkey, mouse, rat, guinea pig, dog, cat, rabbit, pig, cow, horse, goat, and donkey. In various embodiments, the antibody is a humanized antibody. In various embodiments, the antibody is a chimeric antibody. In certain embodiments, the CD105 antagonist is TRC105 or an antigen-binding fragment thereof. In various embodiments, the antigen is CD105. In various embodiments, the antigen is endoglin.

In various embodiments, the cancer has functional p53. In various embodiments, the administration of the CD105 antagonist results in depletion of ATP in the subject with cancer. In various embodiments, the depletion of ATP in cancers with functional p53 results in radiation sensitization. In various embodiments, the CD105 antagonist is an antibody specifically binding to CD105 or an antigen-binding fragment thereof. In various embodiments, the CD105 antagonist is TRC105 or an antigen-binding fragment thereof.

In various embodiments, the sensitization observed by the administration of the CD105 antagonist occurs through a non-vascular mechanism.

In some embodiments, the cancer therapy is surgery. In various embodiments, administering the cancer therapy comprises performing a surgery on the subject. In various embodiments, the surgery removes the cancer. In certain embodiments, the surgery is mastectomy. In certain embodiments, the surgery is orchiectomy (surgical castration).

In some embodiments, the cancer therapy is radiotherapy. In various embodiments, administering the cancer therapy comprises administering a radiation to the subject. In various embodiments, administering the cancer therapy comprises administering a radiotherapeutic agent to the subject. In some embodiments, the CD105 antagonist and the radiotherapeutic agent are provided in a single composition. In other embodiments, the CD105 antagonist and the radiotherapeutic agent are provided in separate compositions.

In various embodiments, the radiotherapy is focused radiotherapy, external beam radiation therapy, conventional external beam radiation therapy (2DXRT), image guided radiotherapy (IGRT), three-dimensional conformal radiation therapy (3D-CRT), intensity modulated radiation therapy (IMRT), helical tomotherapy, volumetric modulated arc therapy (VMAT), particle therapy, proton beam therapy, conformal proton beam radiation therapy, auger therapy (AT), stereotactic radiation therapy, stereotactic radiosurgery (SRS), stereotactic body radiation therapy (SBRT), brachytherapy, internal radiation therapy, intraoperative radiation therapy (TORT), radioimmunotherapy, radioisotope therapy, hyperfractionated radiotherapy, or hypofractionated radiotherapy, or a combination thereof.

Typical dosages of an effective amount of radiation to be administered to the subject can be in the ranges recommended by manufacturer, radiation biologist, radiation oncologist or medical physicist where known radiotherapy techniques are used, and also as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the radiotherapy technique based, for example, on the in vitro responsiveness of relevant cultured cells or histocultured tissue sample, or the responses observed in the appropriate animal models. For example, mice models of pancreatic cancer may be subjected to energy-responsive agent delivery using the SonRx technology and focused radiotherapy using X-RAD small animal irradiator; appropriate parameters for carriers, agents, ultrasound and radiation (e.g., their types, dosages and timing) on the SonRx technology and radiotherapy are identified to maximize clinical outcomes and the therapeutic ratio; and these data serve as basis for translation to clinical trials and treatments in humans. In some embodiments of present invention, typical in vitro and in vivo doses may range from 50 cGy to 8 Gy daily fractions with total treatment doses ranging from 1 Gy to 50 Gy.

In various embodiments, the radiation dosage has a daily treatment dose of about 1-10, 10-20, 20-30, 30-40, 40-50, 50-60, 60-70, 70-80, 80-90, or 90-100 cGy. In various embodiments, the radiation dosage has a daily treatment dose of about 0.1-1, 1-2, 2-3, 3-4, 4-5, 5-6, 6-7, 7-8, 8-9, or 9-10 Gy. In various embodiments, the radiation dosage has a daily treatment dose of about 1-10, 10-20, 20-30, 30-40, 40-50, 50-60, 60-70, 70-80, 80-90, or 90-100 Gy. In various embodiments, the radiation dosage has a total treatment dose of about 0.1-1, 1-2, 2-3, 3-4, 4-5, 5-6, 6-7, 7-8, 8-9, or 9-10 Gy. In various embodiments, the radiation dosage has a total treatment dose of about 1-10, 10-20, 20-30, 30-40, 40-50, 50-60, 60-70, 70-80, 80-90, or 90-100 Gy.

In some embodiments, the cancer therapy is chemotherapy. In various embodiments, administering the cancer therapy comprises administering a chemotherapeutic agent to the subject. In some embodiments, the CD105 antagonist and the chemotherapeutic agent are provided in a single composition. In other embodiments, the CD105 antagonist and the chemotherapeutic agent are provided in separate compositions.

In various embodiments, the cancer therapy does not comprise a tyrosine kinase inhibitor. In various embodiments, the cancer therapy does not comprise axitinib. In various embodiments, the cancer therapy does not comprise pazopanib. In various embodiments, the cancer therapy does not comprise sorafenib.

In some embodiments, the cancer therapy is hormone therapy. In various embodiments, administering the cancer therapy comprises administering a hormone therapeutic agent to the subject. In some embodiments, the CD105 antagonist and the hormone therapeutic agent are provided in a single composition. In other embodiments, the CD105 antagonist and the hormone therapeutic agent are provided in separate compositions. In certain embodiments, the hormone therapeutic agent is enzalutamide. In certain embodiments, the hormone therapeutic agent is abiraterone. In various embodiments, TRC105 and abiraterone are administered to the subject.

In some embodiments, the hormone therapy is an androgen deprivation therapy. In other embodiments, the hormone therapy is an androgen targeted therapy (ATT). In accordance with the present invention, androgen deprivation therapy (ADT, also called androgen suppression therapy) refers to a hormone therapy for treating prostate cancer. Prostate cancer cells usually require androgen hormones, such as testosterone, to grow. ADT reduces the levels of androgen hormones, with drugs or surgery, to prevent the prostate cancer cells from growing. The surgical approaches include orchiectomy (surgical castration). The pharmaceutical approaches include antiandrogens and chemical castration.

In various embodiments, administering the cancer therapy comprises administering a second therapeutic agent to the subject. In some embodiments, the CD105 antagonist and the second therapeutic agent are provided in a single composition. In other embodiments, the CD105 antagonist and the second therapeutic agent are provided in separate compositions. In various embodiments, the second therapeutic agent is a radiotherapeutic agent, chemotherapeutic agent, or a hormone therapeutic agent, or a combination thereof. In some embodiments, the second therapeutic agent is a radiotherapeutic agent. In some embodiments, the second therapeutic agent is a chemotherapeutic agent. In some embodiments, the second therapeutic agent is a hormone therapeutic agent.

In accordance with the present invention, examples of the chemotherapeutic agent include but are not limited to Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Doxifluridine, Doxorubicin, liposome-encapsulated Doxorubicin such as as Doxil (pegylated form), Myocet (nonpegylated form) and Caelyx, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Docetaxel, Cabazitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, prednisone, methylprednisolone, dexamethasone or a combination thereof. In certain embodiments, the chemotherapeutic agent is a taxane. Examples of the taxane include but are not limited to paclitaxel, protein-bound paclitaxel, nab-paclitaxel, docetaxel, and cabazitaxel. In certain embodiments, the chemotherapeutic agent is a vinca alkaloid. Examples of the vinca alkaloid include but are not limited to vinblastine, vincristine, vindesine, and vinorelbine. In certain embodiments, the chemotherapeutic agent is a platinum-based drug. Examples of the platinum-based drug include but are not limited to oxaliplatin, cisplatin, lipoplatin (a liposomal version of cisplatin), carboplatin, satraplatin, picoplatin, nedaplatin, and triplatin. In certain embodiments, the chemotherapeutic agent is a anthracycline. Examples of the anthracycline include but are not limited to doxorubicin, daunorubicin, epirubicin, idarubicin, pirarubicin, aclarubicin, valrubicin, and mitoxantrone. In certain embodiments, the chemotherapeutic agent loaded to the carrier is doxorubicin, or its functional equivalent, analog, derivative, variant or salt, or a combination thereof.

In accordance with the present invention, examples of the hormone therapeutic agent include but are not limited to antiandrogens, VT-464, ODM-201, galeterone, AR antagonists such as flutamide, nilutamide, bicalutamide, enzalutamide, apalutamide (ARN-509), cyproterone acetate, megestrol acetate, chlormadinone acetate, spironolactone, canrenone, drospirenone, ketoconazole, topilutamide (fluridil), cimetidine; selective androgen receptor modulators (SARMs) such as testosterone esters (such as testosterone enanthate, propionate, or cypionate), enobosarm (Ostarine, MK-2866, GTx-024), BMS-564,929, LGD-4033, AC-262,356, JNJ-28330835, LGD-2226, LGD-3303, S-40503, S-23, and andarine (S-4); 5α-reductase inhibitors such as finasteride, dutasteride, alfatradiol, and saw palmetto extract; CYP17A1 (17α-hydroxylase, 17,20-lyase) inhibitors such as cyproterone acetate, spironolactone, danazol, gestrinone, ketoconazole, abiraterone, and abiraterone acetate; 3β-Hydroxysteroid dehydrogenase inhibitors such as danazol, gestrinone, and abiraterone acetate; 17β-Hydroxysteroid dehydrogenase inhibitors such as danazol and simvastatin; CYP11A1 (cholesterol side-chain cleavage enzyme) inhibitors such as aminoglutethimide and danazol; HMG-CoA reductase inhibitors such as statins (e.g., atorvastatin, simvastatin); antigonadotropins, progestogens such as progesterone, cyproterone acetate, medroxyprogesterone acetate, megestrol acetate, chlormadinone acetate, spironolactone, and drospirenone; estrogens such as estradiol, ethinyl estradiol, diethylstilbestrol, and conjugated equine estrogens; GnRH analogues, GnRH agonists such as buserelin, deslorelin, gonadorelin, goserelin, histrelin, leuprorelin, nafarelin, and triptorelin; GnRH antagonists such as abarelix, cetrorelix, degarelix, and ganirelix; anabolic steroids (e.g., nandrolone, oxandrolone); LHRH agonists, LHRH antagonists, leuprolide, goserelin, triptorelin, histrelin, and degarelix. Some agents can act via multiple mechanisms of action, and are hence given as examples in multiple categories.

### Dosage and Administration

Typical dosages of an effective amount of a therapeutic agent as described herein (e.g., CD105 antagonists, radiotherapeutic agents, chemotherapeutic agents and hormone therapeutic agents) can be in the ranges recommended by the manufacturer where known therapeutic molecules or compounds are used, and also as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models. In various embodiments, the therapeutic agent may be administered once a day (SID/QD), twice a day (BID), three times a day (TID), four times a day (QID), or more, so as to administer an effective amount of the therapeutic agent to the subject, where the effective amount is any one or more of the doses described herein.

In various embodiments, a therapeutic agent as described herein (e.g., CD105 antagonists, radiotherapeutic agents, chemotherapeutic agents and hormone therapeutic agents) is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof. In various embodiments, a therapeutic agent as described herein (e.g., CD105 antagonists, radiotherapeutic agents, chemotherapeutic agents and hormone therapeutic agents) is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m², or a combination thereof. In various embodiments, a therapeutic agent as described herein is administered once, twice, three or more times. In some embodiments, a therapeutic agent as described herein is administered 1-3 times per day, 1-7 times per week, 1-9 times per month, or 1-12 times per year. Still in some embodiments, a therapeutic agent as described herein is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. Here, “mg/kg” refers to mg per kg body weight of the subject, and “mg/m²” refers to mg per m² body surface area of the subject.

In various embodiments, the effective amount of a therapeutic agent as described herein (e.g., CD105 antagonists, radiotherapeutic agents, chemotherapeutic agents and hormone therapeutic agents) is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/kg/day, or a combination thereof. In various embodiments, the effective amount of a therapeutic agent as described herein (e.g., CD105 antagonists, radiotherapeutic agents, chemotherapeutic agents and hormone therapeutic agents) is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/m²/day, or a combination thereof. In various embodiments, the effective amount of a therapeutic agent as described herein (e.g., CD105 antagonists, radiotherapeutic agents, chemotherapeutic agents and hormone therapeutic agents) is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg/day, or a combination thereof. In various embodiments, the effective amount of a therapeutic agent as described herein (e.g., CD105 antagonists, radiotherapeutic agents, chemotherapeutic agents and hormone therapeutic agents) is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m²/day, or a combination thereof. Here, “μg/kg/day” or “mg/kg/day” refers to μg or mg per kg body weight of the subject per day, and “μg/m²/day” or “mg/m²/day” refers to μg or mg per m² body surface area of the subject per day.

In some embodiments, a therapeutic agent as described herein (e.g., CD105 antagonists, radiotherapeutic agents, chemotherapeutic agents and hormone therapeutic agents) may be administered at the treatment stage of a cancer (i.e., when the subject has already developed the cancer). In some embodiments, a therapeutic agent as described herein (e.g., CD105 antagonists, radiotherapeutic agents, chemotherapeutic agents and hormone therapeutic agents) may be administered at the maintenance stage of a cancer (i.e., when the subject is in the process to achieve cancer remission). In other embodiments, a therapeutic agent as described herein (e.g., CD105 antagonists, radiotherapeutic agents, chemotherapeutic agents and hormone therapeutic agents) may be administered at the recurrence prevention stage of a cancer (i.e., when the subject has not developed cancer recurrence but is likely to or in the process to develop cancer recurrence).

In various embodiments of the invention, a second therapeutic agent is administered to the subject. In various embodiments, the second therapeutic agent is a radiotherapeutic agent, chemotherapeutic agent, or a hormone therapeutic agent, or a combination thereof. In some embodiments, the second therapeutic agent is a radiotherapeutic agent. In some embodiments, the second therapeutic agent is a chemotherapeutic agent. In some embodiments, the second therapeutic agent is a hormone therapeutic agent.

In various embodiments, the second therapeutic agent in the composition is provided in mg per kilogram body weight of the subject; for example, about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg. In various embodiments, the second therapeutic agent in the composition is provided in mg per m² body surface area of the subject; for example, about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m².

In accordance with the invention, the therapeutic agent as described herein (e.g., CD105 antagonists, radiotherapeutic agents, chemotherapeutic agents and hormone therapeutic agents) may be administered using the appropriate modes of administration, for instance, the modes of administration recommended by the manufacturer for each of the therapeutic agents. In accordance with the invention, various routes may be utilized to administer a therapeutic agent as described herein (e.g., CD105 antagonists, radiotherapeutic agents, chemotherapeutic agents and hormone therapeutic agents). “Route of administration” may refer to any administration pathway known in the art, including but not limited to oral, topical, aerosol, nasal, via inhalation, anal, intra-anal, peri-anal, transmucosal, transdermal, parenteral, enteral, via continuous infusion, or via an implantable pump or reservoir or local administration. “Parenteral” refers to a route of administration that is generally associated with injection, including intratumoral, intracranial, intraventricular, intrathecal, epidural, intradural, intraorbital, intraocular, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the agent or composition may be in the form of solutions or suspensions for infusion or for injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions or emulsions. Typically, the compositions are administered by injection. Methods for these administrations are known to one skilled in the art.

In an embodiment, agent or composition may be provided in a powder form and mixed with a liquid, such as water, to form a beverage. In accordance with the present invention, “administering” can be self-administering. For example, it is considered as “administering” that a subject consumes a composition as disclosed herein. In various embodiments, a therapeutic agent as described herein (e.g., CD105 antagonists, radiotherapeutic agents, chemotherapeutic agents and hormone therapeutic agents) is administered intracranially, intraventricularly, intrathecally, epidurally, intradurally, topically, intratumorally, intravascularly, intravenously, intraarterially, intramuscularly, subcutaneously, intraperitoneally, intranasally, orally, intraorbitally, or intraocularly.

In various embodiments, the CD105 antagonist is an antibody specifically binding to CD105 or an antigen-binding fragment thereof. In some embodiments, the antibody is a polyclonal antibody. In other embodiments, the antibody is a monoclonal antibody. In various embodiments, the antibody can be of any animal origin. Examples of the animal origin include but are not limited to human, non-human primate, monkey, mouse, rat, guinea pig, dog, cat, rabbit, pig, cow, horse, goat, and donkey. In various embodiments, the antibody is a humanized antibody. In various embodiments, the antibody is a chimeric antibody. In certain embodiments, the CD105 antagonist is TRC105 or an antigen-binding fragment thereof.

In various embodiments, the CD105 antagonist in the composition is provided in mg per kilogram body weight of the subject; for example, about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg. In various embodiments, the CD105 antagonist in the composition is provided in mg per m² body surface area of the subject; for example, about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m².

Preferred therapeutic agents will also exhibit minimal toxicity when administered to a mammal.

In various embodiments, the composition is administered once, twice, three or more times. In various embodiments, the composition is administered 1-3 times per day, 1-7 times per week, 1-9 times per month, or 1-12 times per year. In various embodiments, the composition is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In various embodiments, the composition may be administered once a day (SID/QD), twice a day (BID), three times a day (TID), four times a day (QID), or more, so as to administer an effective amount of the CD105 antagonist and the second therapeutic agent to the subject, where the effective amount is any one or more of the doses described herein.

In various embodiments, the therapeutic agent according to the invention can contain any pharmaceutically acceptable excipient. As used herein, an “excipient” is a natural or synthetic substance formulated alongside the active ingredient of a composition or formula, included for the purpose of bulking-up the composition or formula. Thus, “excipient” is often referred to as “bulking agent”, “filler”, or “diluent”. For a non-limiting example, one or more excipients may be added to a therapeutic agent described herein and increase the composition's volume or size so that one serving of the composition fits into one capsule or tablet. Also, an “excipient” may confer an enhancement on the active ingredients in the final dosage form, such as facilitating absorption or solubility of the active ingredients. “Pharmaceutically acceptable excipient” means an excipient that is useful in preparing a therapeutic agent that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the therapeutic agent can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The therapeutic agent can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The therapeutic agents are made following the conventional techniques of pharmacy involving dry milling, mixing, and blending for powder forms; milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The therapeutic agent may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

Polymers formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268, 110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid therapeutic agent is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The therapeutic agent may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

### Kits of the Invention

In various embodiments, the present invention provides a kit for sensitizing a cancer in a subject. The kit comprises: a quantity of a CD105 antagonist; and instructions for using the CD105 antagonist to sensitize the cancer. In various embodiments, the cancer is sensitized to a cancer therapy.

In various embodiments, the present invention provides a kit for treating, slowing the progression of, reducing the severity of, preventing the recurrence of, and/or reducing the recurrence likelihood of a cancer in a subject. The kit comprises: a quantity of a CD105 antagonist; a cancer therapy; and instructions for using the CD105 antagonist and the cancer therapy to treat, to slow the progression of, to reduce the severity of, to prevent the recurrence of, and/or to reduce the recurrence likelihood of the cancer in the subject.

In various embodiments, the present invention provides a kit for preventing the recurrence of and/or reducing the recurrence likelihood of a cancer in a subject. The kit comprises: a quantity of a CD105 antagonist; and instructions for using the CD105 antagonist to prevent the recurrence of and/or to reduce the recurrence likelihood the cancer. In various embodiments, the subject has been treated with a cancer therapy.

In various embodiments, the CD105 antagonist is an antibody specifically binding to CD105 or an antigen-binding fragment thereof. In some embodiments, the antibody is a polyclonal antibody. In other embodiments, the antibody is a monoclonal antibody. In various embodiments, the antibody can be of any animal origin. Examples of the animal origin include but are not limited to human, non-human primate, monkey, mouse, rat, guinea pig, dog, cat, rabbit, pig, cow, horse, goat, and donkey. In various embodiments, the antibody is a humanized antibody. In various embodiments, the antibody is a chimeric antibody. In certain embodiments, the CD105 antagonist is TRC105 or an antigen-binding fragment thereof.

In various embodiments, the cancer therapy is radiotherapy, chemotherapy, hormone therapy, or surgery, or a combination thereof.

In some embodiments, the cancer therapy is surgery. In various embodiments, the kit comprises equipment, tools, materials and instructions for performing a surgery on the subject. In various embodiments, the surgery removes the cancer. In certain embodiments, the surgery is mastectomy. In certain embodiments, the surgery is orchiectomy (surgical castration).

In some embodiments, the cancer therapy is radiotherapy. In various embodiments, the kit comprises equipment, tools, materials and instructions for administering a radiotherapy to the subject. In various embodiments, the kit comprises a quantity of a radiotherapeutic agent and instructions for using the radiotherapeutic agent to treat, to slow the progression of, to reduce the severity of, to prevent the recurrence of, and/or to reduce the recurrence likelihood of the cancer in the subject. In some embodiments, the CD105 antagonist and the radiotherapeutic agent are provided in a single composition. In other embodiments, the CD105 antagonist and the radiotherapeutic agent are provided in separate compositions.

In some embodiments, the cancer therapy is chemotherapy. In various embodiments, the kit comprises a quantity of a chemotherapeutic agent and instructions for using the chemotherapeutic agent to treat, to slow the progression of, to reduce the severity of, to prevent the recurrence of, and/or to reduce the recurrence likelihood of the cancer in the subject. In some embodiments, the CD105 antagonist and the chemotherapeutic agent are provided in a single composition. In other embodiments, the CD105 antagonist and the chemotherapeutic agent are provided in separate compositions.

In some embodiments, the cancer therapy is hormone therapy. In various embodiments, the kit comprises a quantity of a hormone therapeutic agent and instructions for using the hormone therapeutic agent to treat, to slow the progression of, to reduce the severity of, to prevent the recurrence of, and/or to reduce the recurrence likelihood of the cancer in the subject. In some embodiments, the CD105 antagonist and the hormone therapeutic agent are provided in a single composition. In other embodiments, the CD105 antagonist and the hormone therapeutic agent are provided in separate compositions.

The kit is an assemblage of materials or components, including at least one of the inventive compositions or components. Thus, in some embodiments the kit contains a composition including a drug delivery molecule complexed with a therapeutic agent as described above.

The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome. Optionally, the kit also contains other useful components, such as, containers, spray bottles or cans, diluents, buffers, pharmaceutically acceptable carriers, syringes, catheters, applicators (for example, applicators of intravenous infusion, cream, gel or lotion etc.), pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in assays and therapies. As used herein, the term “package” refers to a suitable solid matrix or material such as glass, plastic, paper, foil, and the like, capable of holding the individual kit components. Thus, for example, a package can be a preloaded syringe, preloaded injection pen, or glass vial containing suitable quantities of a composition as described herein. The packaging material generally has an external label which indicates the contents and/or purpose of the kit and/or its components.

Many variations and alternative elements have been disclosed in embodiments of the present invention. Still further variations and alternate elements will be apparent to one of skill in the art. Among these variations, without limitation, are the selection of constituent modules for the inventive methods, compositions, kits, and systems, and the various conditions, diseases, and disorders that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” As one non-limiting example, one of ordinary skill in the art would generally consider a value difference (increase or decrease) no more than 5% to be in the meaning of the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### EXAMPLES

The invention will be further explained by the following Examples, which are intended to be purely exemplary of the invention, and should not be considered as limiting the invention in any way. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1 Extra-Vascular Actions of TRC105 in Prostate Cancer Castrate Resistance

Using FACS analysis on carcinoma associated fibroblasts from prostate cancer tissues, we identified CD105 to be an important factor that defines tumor inductivity. CD105 expression was elevated in carcinoma in carcinoma prostatic associated fibroblasts over their non-cancerous counterpart, and increased with radiation exposure. TRC105 is a blocking antibody for CD105, preventing the binding of its cognate ligands. TRC105 was used on prostate fibroblasts to test signaling activity by Western blotting.

As the stromal support of cancer is important to its progression, we believe that antagonizing CD105 signaling can inhibit cancer progression through its action in both endothelial cells and cancer associated fibroblasts. In FIG. 7, blocking endogenous host vasculature with M1043 (mouse specific CD105 antagonist) effectively reduced tumor vascularity, but had no significant effect on tumor size in the context of ATT.

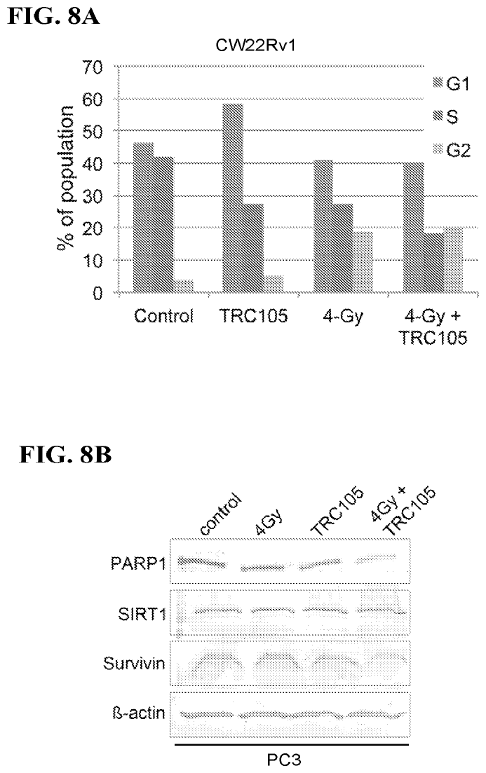

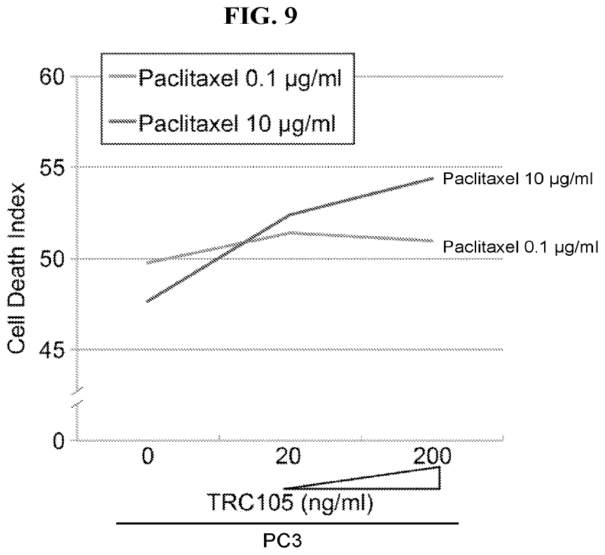

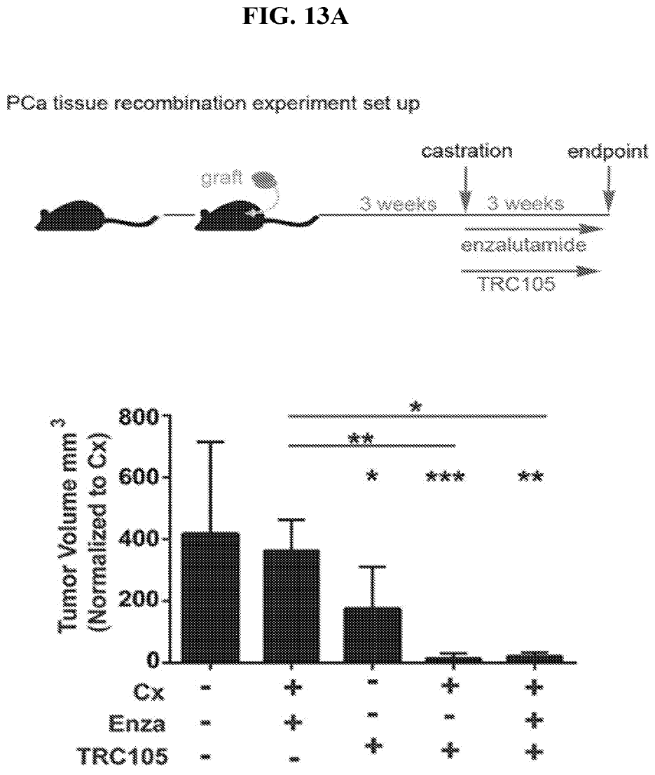

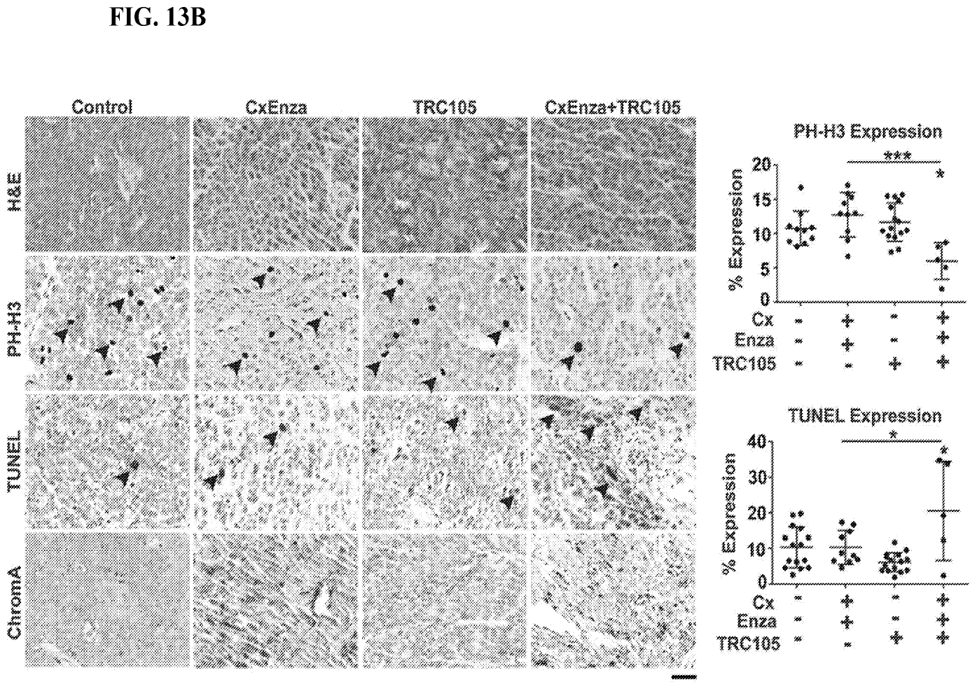

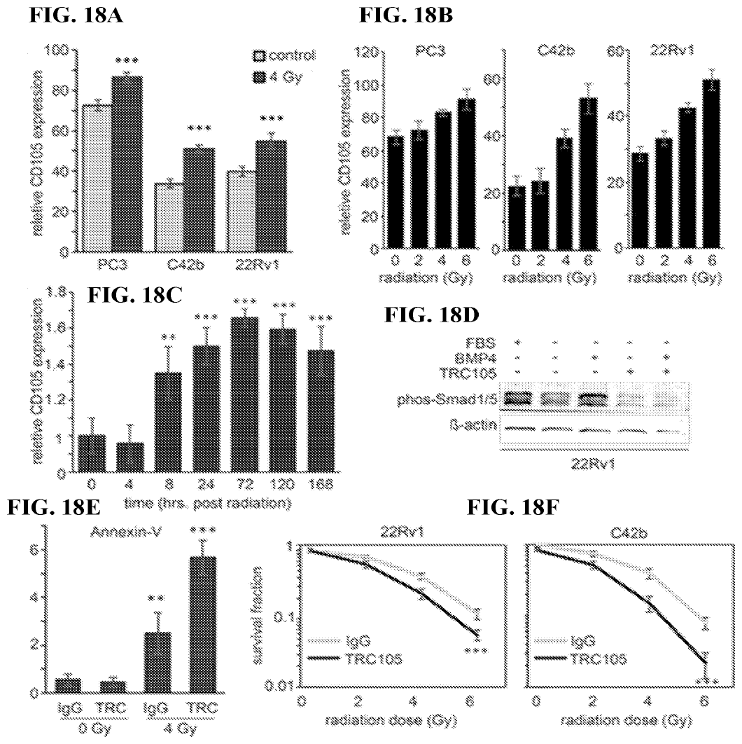

TRC105 is tested for complementing radiation treatment of tumor models in vitro and in vivo (see FIGS. 8A, 8B and 18A). Preclinical testing has also been performed in prostate cancer models using a combination of TRC105 and docetaxel (see FIG. 9). In addition, TRC105 treatment in castrated mice grafted with a prostate cancer epithelia xenograft reduced tumor size (see FIG. 13). TRC105 was also combined with enzalutamide as another way of inhibiting androgen signaling, and yielded similar results (see FIG. 13).

Antagonizing CD105 in the prostatic stromal fibroblasts can mediate castration sensitivity, while antagonizing CD105 in the prostatic vasculature has limited therapeutic value. There is a safe and tolerable combination of abiraterone or enzalutamide with TRC105. Inhibition of CD105 can overcome resistance to AR-targeted therapy inducing clinical benefit in patients who have developed early resistance to AR-targeted therapy. In clinical trial design, EWOC (Escalation With Overdose Control—a Bayesian adaptive drug dose finding design) combinatorial phase I is to develop MTD (maximum tolerated dose) curve. Abiraterone dosing: 500, 750, 1000 mg qd; enzalutamide dosing 80, 120, 160 mg qd; and TRC105 dosing: 10-20 mg/kg (continuous variable). Eligibility is for patients currently on either abiraterone or enzalutamide who are showing early signs of progressing by rising PSA ECOG PS 0-1.

### Example 2 Androgen-Dependent Alteration of the Heterogeneous CAF Population Potentiates Castrate Resistance

### Stromal Cell Heterogeneity Maintains Tumor Promoting Capacity

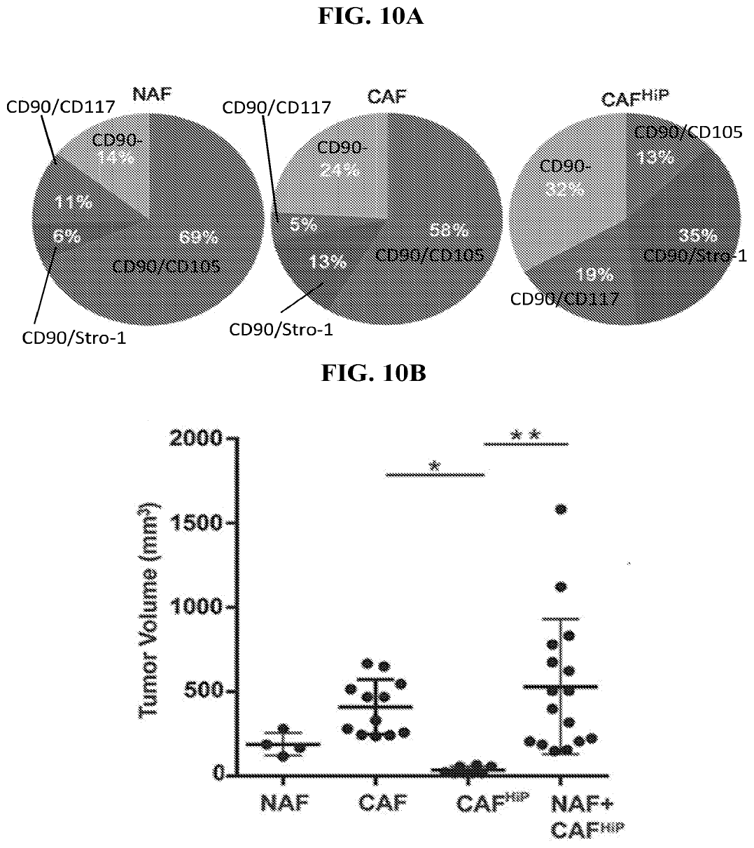

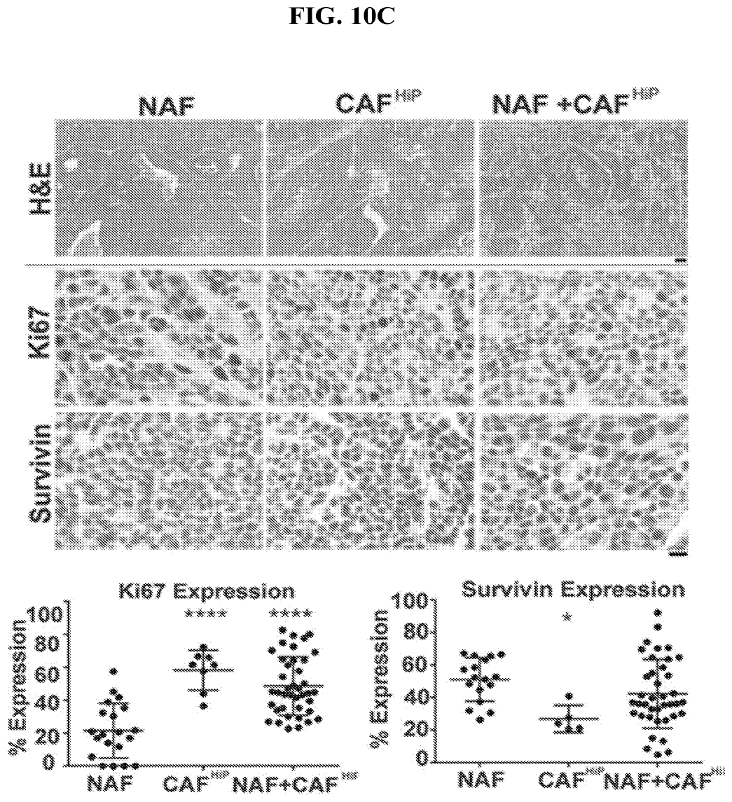

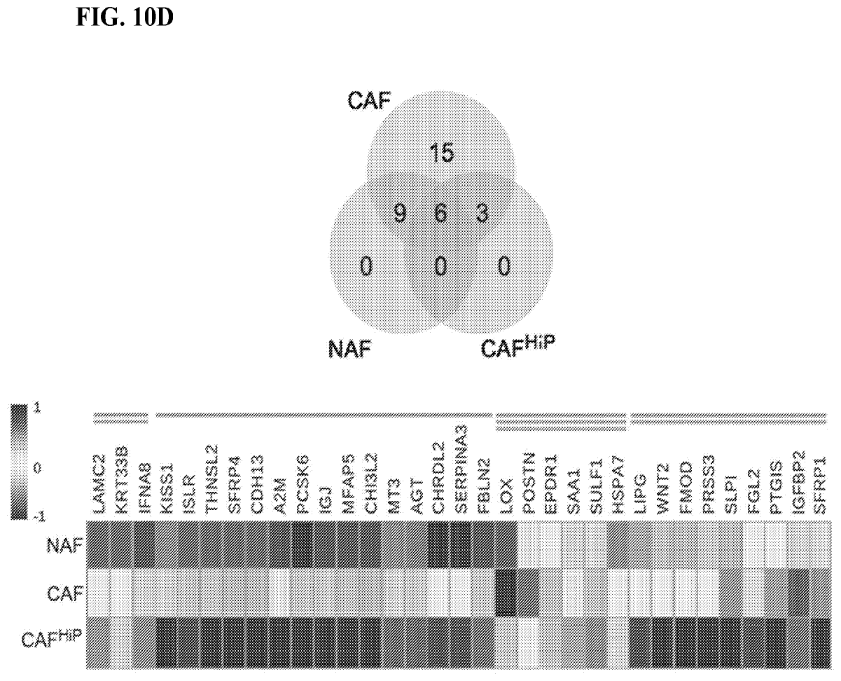

Primary CAF cultures generated from prostatectomy tissues can promote the expansion of established tumor epithelia. However, it was observed that routine culturing of the primary PCa CAFs can lead to the loss of tumor promoting capacity. The inventors compared low passage CAF (between 3-7 passage) to high passage (CAF^{HiP}, >8 passage) by cell surface expression of CD90, CD105, CD117, and STRO-1 in the NAF, CAF, and CAF^{HiP} (FIG. 10A). Other markers, PDGF receptor, CD133, and CD10 were also tested, but were not found to be differentially expressed among the stromal cell types. The CD117⁺/CD90⁺ population was down-regulated in the tumor-inductive CAF, compared to either the non-inductive CAF^{HIP} and NAF. The Stro-1⁺/CD90⁺ fibroblastic population was elevated in CAF compared to NAF, yet further elevated in the CAF^{HiP}. Interestingly, the most abundant fibroblastic population in both NAF and CAF was the CD90⁺/CD105⁺ population, found to be depleted in the CAF^{HiP}.

Without being bound to any particular theory, it is believed that prolonged culturing, results in the loss of stromal heterogeneity. Thus, the inventors attempted to unsuccessfully deplete the CD105, CD117, and Stro-1 populations in CAF by flow sorting. Interestingly, the expansion of the sorted cultures past seven days revealed a restoration of the original stromal cell surface-marker distribution (data not shown). Accordingly, restoration of the depleted CD90⁺/CD105⁺ population in CAF^{HiP} was achieved by adding NAF cells through the generation of tissue recombination xenografts using human CWR22Rv1 (Rv1) PCa epithelia. Strikingly, the combination of two non-tumor inductive stromal populations, CAF^{HiP}/NAF, gave rise to tumors that were larger than those with either NAFs or CAF^{HiP} alone, statistically similar to CAF (FIG. 10B). Histologic analysis of the tumors revealed that epithelial proliferation was significantly elevated in CAF^{HiP} and the CAF^{HiP}/NAF stromal cell combination compared to NAF, as determined by Ki67 immunohistochemistry (FIG. 10C). However, there was a significant decrease of epithelial survivin expression in CAF^{HiP}-associated tumors, compared to NAF or CAF recombinant tumors. Although somewhat counterintuitive, the loss of tumor promoting capacity in CAF^{HiP} could be restored with the addition of non-tumorigenic NAFs.

To identify the differences in paracrine mediators among the three stromal cell types, the inventors performed RNA-sequencing and segregated the genes based on their expression pattern. Differential gene expression among CAFs, CAF^{HiP}, and NAFs was analyzed based on the combined ranked ratios of CAF/CAF^{HiP} and CAF/NAF. Candidate paracrine mediators (secreted genes, by gene ontology analysis from the top 200 differentially regulated genes) were plotted in a Venn diagram, revealing 9 genes expressed in both NAF and CAF not found in the CAF^{Hip} (FIG. 10D). Conversely, 3 genes were shared by CAF and CAF^{Hip} cells. Presumably, paracrine mediators in the CAF, found differentially expressed in CAF^{Hip} or NAF, enabled the observed tumor expansion.

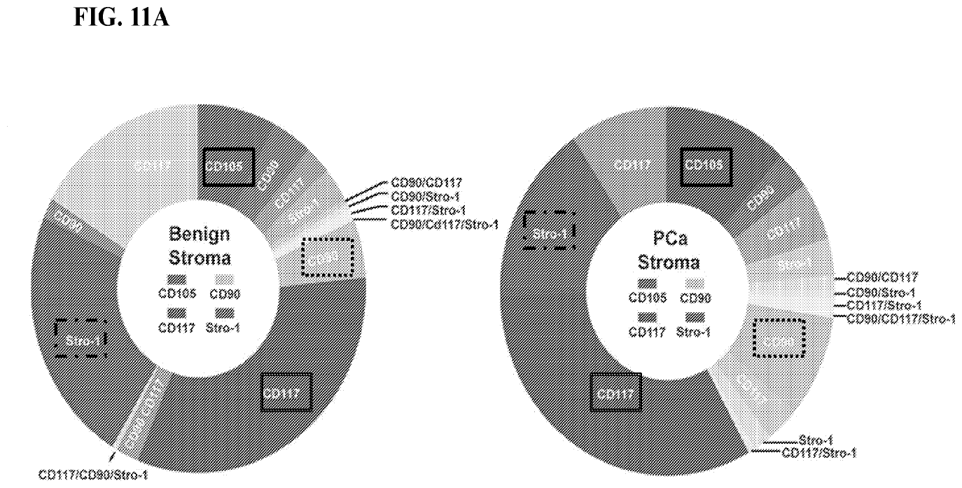

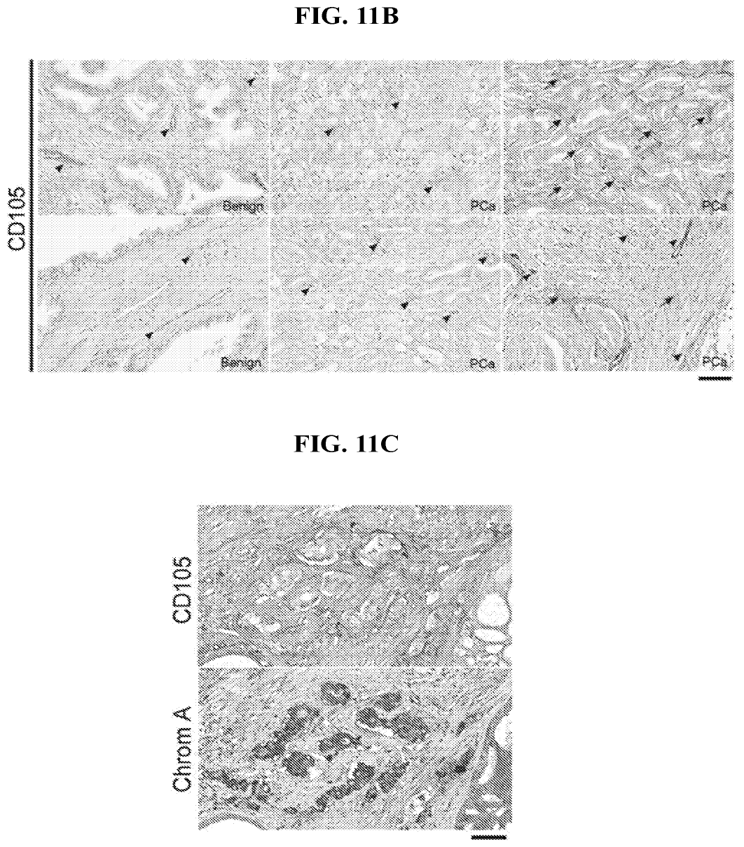

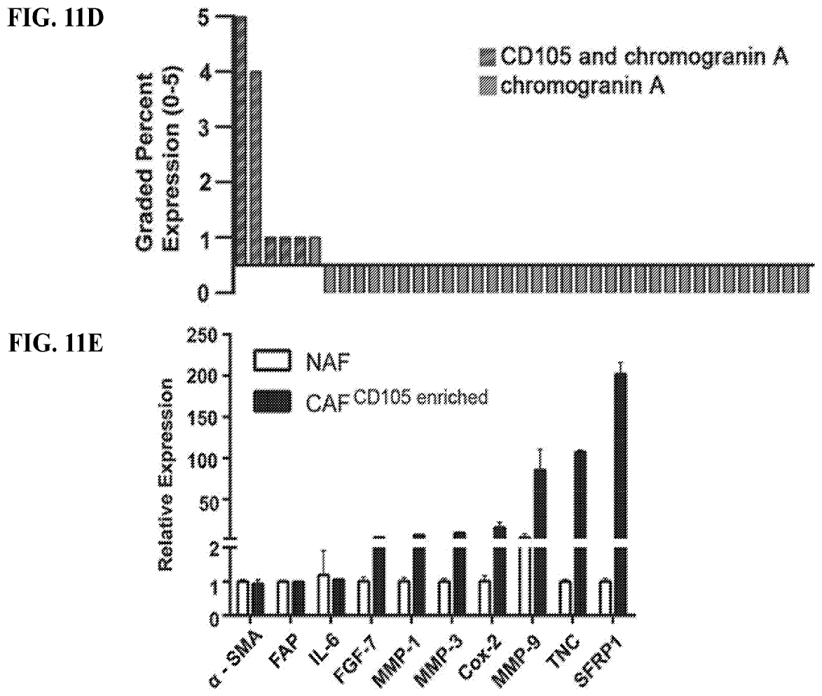

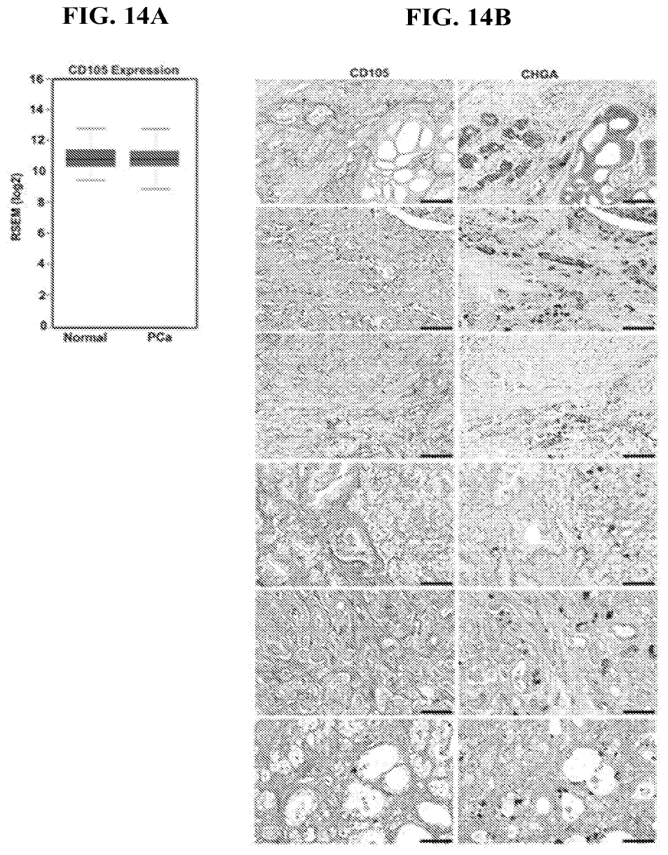

The inventors examined the changes in stromal heterogeneity in prostate stromal populations directly from fresh prostatectomy tissues. Tissues verified by H&E staining from frozen sections to be cancer or benign were dissociated and sorted for the expression of CD90, CD105, CD117, and Stro-1 within the stromal population of four patients. FIG. 11A illustrates the distribution of the cell surface markers, based on the most abundant marker per population with the co-expressed markers adding to the diversity of the individual populations. The CD117-dominant population was the most abundant in both benign and PCa tissues. The Stro-1-dominant population was enriched in stroma from benign tissues. The CD105-dominant population was differentially elevated in the PCa stroma compared to benign tissues. Its expression was validated in 79 PCa and 16 benign tissues by immune-localization in a tissue array. In benign prostate tissues, CD105 staining was primarily restricted to endothelial cells (FIG. 11B). In PCa however, CD105 was expressed in the endothelia and heterogeneously expressed in stromal fibroblastic cells. A correlation of Gleason grade to the expression of stromal fibroblastic CD105 was not observed. Query of the TCGA Research Network did not reveal any differences in CD105 expression in benign and PCa tissues, but CD105 gene amplification was prominently associated with neuroendocrine PCa in the Trento/Cornell/Broad 2016 data (Cerami et al., Cancer Discov. 2012, 2, 401-404; Gao et al., cBioPortal. Sci Signal, 2013, 6, phi), with a hazard ratio >3 (p <0.001, FIG. 14). Interestingly, staining for the neuroendocrine marker, chromogranin A, revealed its expression circumscribed by CD105⁺ fibroblasts (FIGS. 11C and 14). Stromal CD105 was expressed in 83% of tissues with NED, where receiver operating characteristic (ROC) analysis provided an area under the curve (AUC) of 0.751 (p=0.0026, FIG. 11D). Next, a panel of genes were measured to help define the CAF population. MMP-9, tenascinC, and SFRP1 (Secreted Frizzled Related Protein 1) were elevated more than 25-fold in a primary CAF line, enriched in CD105 expression, compared to NAF (FIG. 11E). The traditional markers of alpha-smooth muscle actin, fibroblast activating protein (FAP), and IL-6 were interestingly not especially elevated in the CD105-enriched CAF compared to that in cultured NAF. Together, stromal fibroblastic CD105 expression was associated with PCa epithelia, but highly correlated with NED.

### Role of CD105 in PCa Neuroendocrine Differentiation

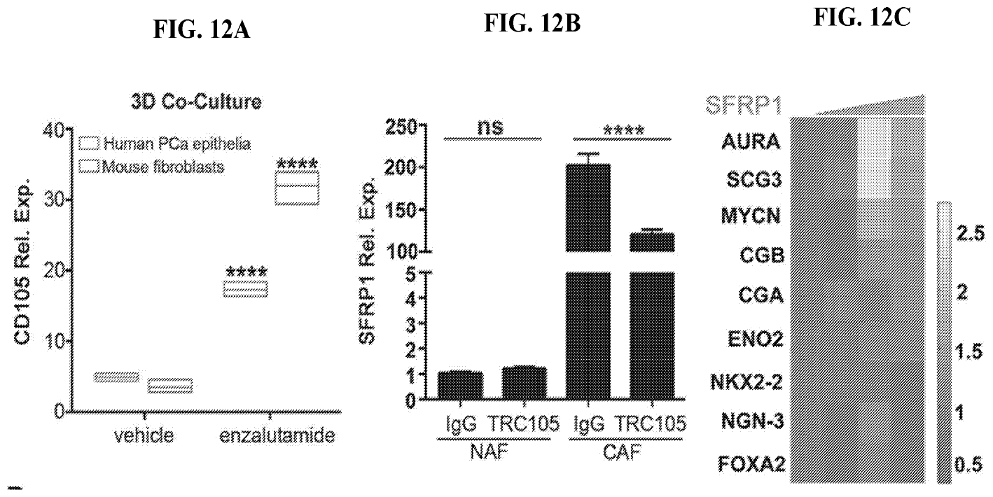

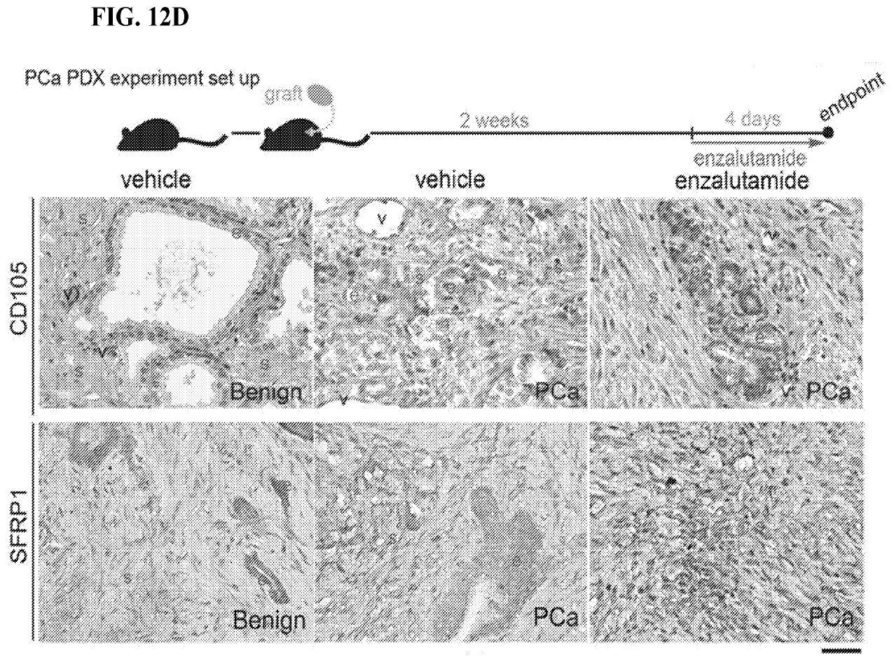

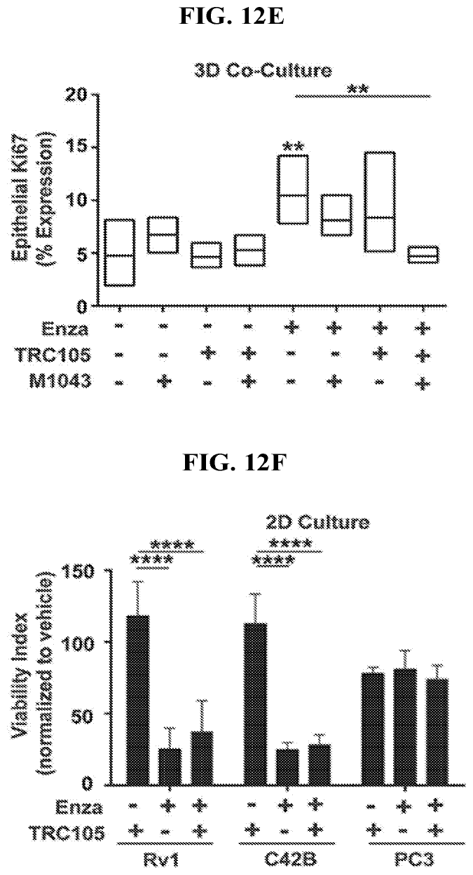

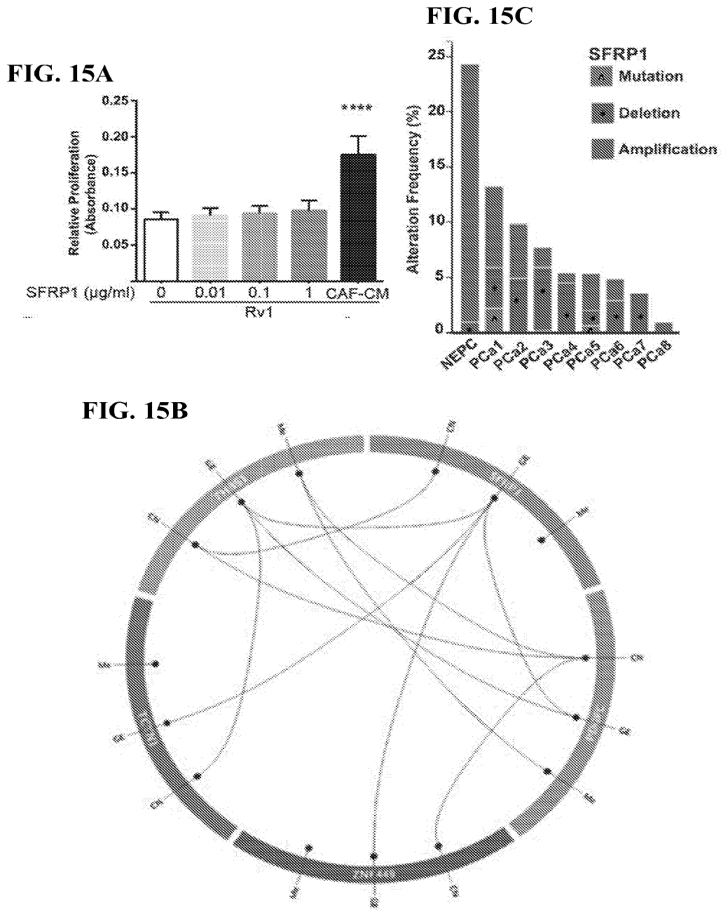

Antagonizing the androgen axis with enzalutamide and/or castration therapy are routine interventions for advanced PCa that eventually lead to neuroendocrine differentiation (NED). To quantitate the cell surface expression of CD105 induced by enzalutamide, the inventors generated 3D cultures with human Rv1 and mouse wild type prostatic fibroblasts. The treatment of these cultures with enzalutamide resulted in a striking three-fold increase in CD105 cell surface expression in epithelial and fibroblastic populations by FACS analysis, compared to vehicle (FIG. 12A). Based on the differential expression of SFRP1 in NAF, CAF, and CAF^{HiP} populations and correlation of CD105 and SFRP1 expression observed, the inventors tested the role of CD105 on SFRP1 expression. The inventors treated NAF and CAF with a CD105 neutralizing antibody, TRC105. SFRP1 expression in CAF was significantly down regulated by TRC105 (p<0.0001), with no effect on NAF. A circus plot in FIG. 15 illustrates the association of SFRP1 gene expression with the expression of thrombospondin 1 (THBS1), platelet derived growth factor 1 (PDGFC), tectonic family member 1 (TCTN1), and zinc finger protein 449 (ZFN449), based on TCGA gene association query. Of the four SFRP1 regulated genes, PDGFC, sonic hedgehog (target of TCTN1), and THBS1 are associated with tumor NED. There was further evidence of the role of SFRP1 in NED of PCa in the TCGA, where its amplification was associated with NED in the Trenton/Cornell/Broad 2016 study (FIG. 15) (Cerami et al., Cancer Discov. 2012, 2, 401-404; Gao et al., cBioPortal. Sci Signal, 2013, 6, p 11). To test the role of SFRP1 on epithelia, the inventors treated cultured Rv1 with recombinant SFRP1 to find significant induction of a panel of 9 PCa NED genes in a dose dependent manner (p<0.002; FIG. 12C). However, the same doses of SFRP1 had no effect on epithelial proliferation (FIG. 15). PCa patient-derived xenograft (PDX) models were examined as a consequence of enzalutamide treatment. Immune-localization of CD105 was predominantly expressed in the vascular endothelia in benign and cancer tissue grafts in the absence of enzalutamide treatment. As with the 3D culture, following 4 days of enzalutamide administration, CD105 expression was elevated in both epithelial and CAF populations in the PDX tissues (FIG. 12D). Concomitantly, SFRP1 was found to be upregulated in the PDX associated with enzalutamide treatment. Thus, without being bound to any particular theory, blocking the androgen axis is associated with CD105, and in turn SFRP1 expression, contributing to NED of PCa.

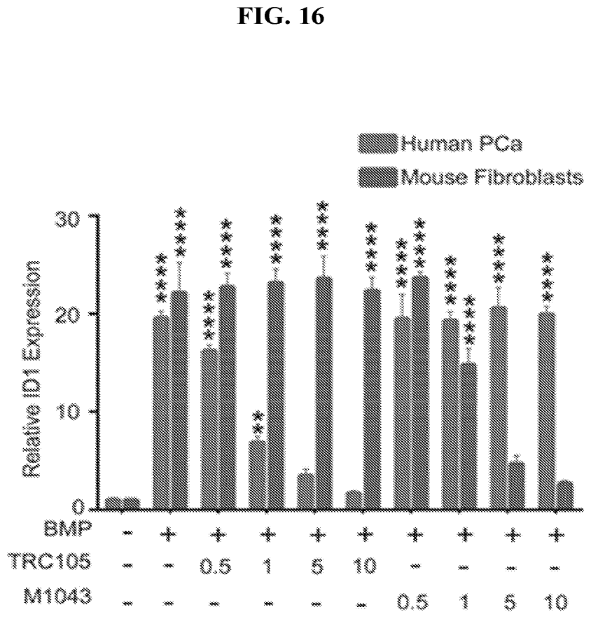

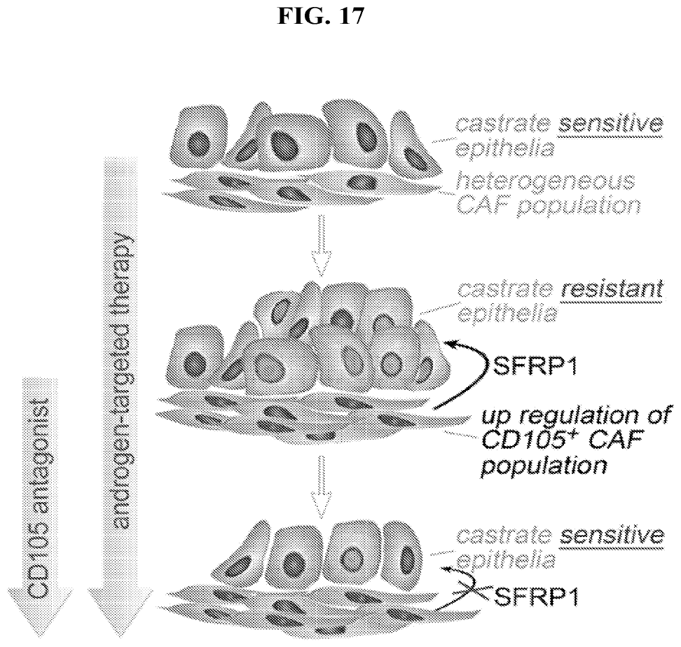

To test if enzalutamide-induced expansion of the CD105⁺ CAF population was consequential to the efficacy of ATT, the inventors generated 3D co-cultures, as described above, with PCa epithelia and stroma with human CW22Rv1 cells and wild type mouse prostatic fibroblasts. In this instance, the species differences allowed targeting of either the epithelia with the human-specific CD105 neutralizing antibody, TRC105, or fibroblasts with the mouse-specific CD105 neutralizing antibody, M1043. At doses used for this study (1 μg/ml), no cross-species reactivity of the two antibodies were found (FIG. 16). The co-cultures were treated with enzalutamide in the presence or absence of TRC105 and/or M1043. The resultant changes in epithelial proliferation were quantitated by FACS analysis of EpCam and Ki-67 co-staining. Treatment with either M1043 or TRC105 alone did not change epithelial proliferation compared to IgG control. In the 3D cultures, CW22Rv1 proliferation index doubled with enzalutamide treatment compared to vehicle (FIG. 12E, p <0.01). Blocking either fibroblastic or epithelial CD105, with M1043 or TRC105, respectively did not alter enzalutamide-induced epithelial proliferation; however, combining enzalutamide with both M1043 and TRC105 restored epithelial proliferation to that of control. The treatment of either Rv1 or C42B (in the absence of fibroblastic cells) with enzalutamide alone significantly reduced cell proliferation, as determined by MTT assay (p<0.0001, FIG. 12F). The addition of TRC105 to enzalutamide provided no additional impact on the proliferation of the PCa epithelia. The PC3 cells, with no androgen receptor expression, were insensitive to either enzalutamide in the presence or absence of TRC105. Thus, the stromal response to enzalutamide was consequential to epithelial proliferation.

### Antagonizing CD105 Sensitizes Castrate Resistant Prostate Cancer to Androgen Targeted Therapy

To determine if antagonizing CD105 sensitizes castrate resistant prostate cancer (CRPC) to androgen targeted therapy (ATT), the inventors tested tissue recombination orthotopic xenografts of primary human CAF and Rv1. The tumors were expanded for 3 weeks prior to castrating the mice and treating with enzalutamide, in the presence or absence of TRC105 for an additional 3 weeks. The castrate resistant tumor recombinant model in the castrated mice given enzalutamide had tumor volumes and histologic measures for cell turnover by phosphorylated-histone H3 and TUNEL localization were statistically comparable to control intact mice (FIG. 13A, 13B). Mice treated with TRC105 alone had tumors smaller than vehicle (p<0.05), yet little change in either proliferation or cell death was observed compared to control. However, the combination of TRC105 in enzalutamide treated castrated mice, resulted in a significant reduction in tumor volume compared to either vehicle or enzalutamide (p<0.01 and <0.05, respectively). The combination of castration, enzalutamide, and TRC105 substantially reduced proliferation compared to either control or castrated enzalutamide treatment group (p<0.05 and <0.001, respectively) and increased TUNEL staining compared to control or enzalutamide treatment (p<0.05). NED elevated by ATT, associated with chromogranin A staining, was reduced by the additional administration of TRC105. Without being bound to any particular therapy, the observed increase in CD105 and NED of PCa associated with antagonizing the androgen axis can be exploited by neutralizing CD105 to limit NED and the provided a means of restoring castrate sensitivity.

### Experimental Procedures

3D organotypic co-culture: A modified version of the 3D organotypic co-culture system was performed in a collagen matrix similar to that previously reported (Stark et al., 1999, J Invest Dermatol 112, 681-691). Collagen matrix gels were prepared by mixing five volumes of rat tail collagen I with two volumes of matrigel (NCO, one volume of 10× DMEM medium (GE Healthcare Life Sciences), and one volume of FBS (Atlanta Biologicals), Rv1 and primary mouse prostatic fibroblasts were combined in a 1:3 ratio. Nylon squares were coated with collagen and placed on metal grids in a 6-well plate. Gel plugs (150 μl) were transferred onto the nylon squares and media was added to the level of the nylon mesh. After 72 hours, the cells were dissociated from the matrix with collagenase and dispersed for FACS analysis.
FACS analysis: FACS experiments were performed with eBiosciences antibodies: anti-human Stro-1-FITC (340105), anti-human CD90-PE (12-0909-42), anti-human CD105-APC (17-1057-41), anti-mouse CD105-APC (17-1051-82), anti-human CD117-PE-Cy7 (15-1178-41), anti-human Ki67-PECy7 (25-5699-41), and anti-human EpCAM-PE (12-9326-41). All events were acquired on a BD LSRII flow cytometer in the Cedars-Sinai Medical Center FACS core installed with BD FACSDiva software. Files were analyzed using FlowJo software v10.2. EpCAM positive cells were used to identify the CW22Rv1 epithelia in three-dimensional (3D) co-cultures and further gated for CD105 or Ki67 positive cells.
Animal studies: Male beige/SCID mice (Envigo), 6-8 or 10-12 weeks old, were used for sub-renal capsule or prostatic orthotopic grafting, respectively, as previously described (Banerjee et al., 2014, Oncogene 33, 4924-4931; Hayward et al., 2001, Cancer Res 61, 8135-8142). In accordance with institutional animal care and use committee approval, 2×10⁵ CW22Rv1 cells and 6×10⁵ stromal cells were suspended in 20 μL type I collagen to be grafted into the subrenal capsule of mice castrated after seven days and sacrificed 21 days after castration. For orthotopic xenografts, mice were castrated after three weeks, treated 3 times weekly with enzalutamide (1 mg/mouse oral gavage) and/or TRC105 (50 μg/mouse i.v.) and sacrificed 21 days after castration. Tumor volume was calculated using the modified ellipsoid formula volume3=π/6×(width)2×length.
Cell lines and culture: CW22Rv1, C42B, and PC3 cells from ATCC were expanded as directed. Prostatectomy specimens from Cedars-Sinai Medical Center or the Greater Los Angeles Veterans Affairs were cultured to generate NAF and CAF cells using the same method, as C57BL/6 mouse wild-type prostatic fibroblasts were grown as previously reported (Franco et al., 2011, Cancer research 71, 1272-1281; Kiskowski et al., 2011, Cancer research 68, 4709-4718). TRC105 and M1043 were provided by TRACON Pharmaceuticals, Inc. (San Diego, Calif.). Enzalutamide (Xtandi) was purchased from Medivation (San Francisco, Calif.). Cells were treated with TRC105 or M1043 (1 μg/mL) and enzalutamide (5 μM), for 72 hours.
CAF conditioned media: CAF were plated at a density to reach confluence by 72 hours in normal culture media as previously reported (Franco et al., 2011, Cancer research 71, 1272-1281; Qi et al., 2013, Cancer cell 23, 332-346). After 72 hours the media was collected, centrifuged to remove cell debris, and supernatant was used fresh or stored at −80oC. Target cells were treated with 50% conditioned media in combination with 50% control media.
Histopathology and immunohistochemistry: Paraffin embedded tissues were sectioned (5 μm thick) were subjected to hematoxylin and eosin (H&E) staining and immunohistochemistry as previously reported (Placencio et al., 2008, Cancer research 68, 4709-4718). Serial sectioned tissue arrays of prostate cancer tissue arrays were purchased from US Biomax, Inc. (Derwood, Md.). Anti-phosphorylated histone H3 (06-570, Millipore), anti-CD105 (NCL-CD105, Leica Microsystems), anti-Ki67 (ab16667, Abcam), and anti-chromogranin A (sc-13090, Santa Cruz Biotechnology), anti-SFRP1 (601-401-475S, Rockland Immunochemicals), and anti-survivin (2808, Cell Signaling) antibodies were incubated at 4° C. overnight. Secondary antibody development was performed with Dako Cytomation mouse or rabbit kits and visualized using 3,3′-diaminobenzidine tetrahydrochloride substrate. TUNEL staining was performed per manufacturer's protocol (S7100, Millipore). Slides were scanned with a Leica Biosystems Aperio AT. Up to five fields per tissue were quantitated with Fiji (ImageJ) using a custom written macro. Mitotic and death index were quantitated by taking the total number of positively-stained nuclei divided by the total number of nuclei.
RNA analysis: Total RNA was extracted using the RNeasy kit (Qiagen). 1 μg RNA was used for cDNA synthesis using iSCRIPT cDNA synthesis kit (1708891, Bio-Rad). Quantitative RT-PCR was performed with 5 replicates using the Step One Real-Time PCR system (Applied Biosystems). Gene mRNA expression was normalized to GAPDH. Primer sequences can be found in the Table 1. For RNA-sequencing, Ion Proton AmpliSeq Transcriptome RNA Sequencing was performed achieving an average of 3M reads. We mapped an average of 88% of the reads to the human genome with Torrent Suite version 4.4.2.

TABLE 1
Primer Sequences

| Gene | Sequence | SEQ ID NO |
|---|---|---|
| SIRT1 Forward | 5′-TGC TGG CCT AAT AGA GTG GCA AAG-3′ | 1 |
| SIRT1 Reverse | 5′-GGC ATG TCC CAC TAT CAC TGT-3′ | 2 |
| ID1 Forward | 5′-AAT CAT GAA AGT CGC CAG TG-3′ | 3 |
| ID1 Reverse | 5′-ATG TCG TAG AGC AGC ACG TTT-3′ | 4 |
| NRF1 Forward | 5′-CAG CAG GTC CAT GTG GCT ACT-3′ | 5 |
| NRF1 Reverse | 5′-GCC GTT TCC GTT TCT TTC C-3′ | 6 |
| MTFA Forward | 5′-GAT GCT TAT AGG GCG GAG TGG-3′ | 7 |
| MTFA Reverse | 5′-GCT GAA CGA GGT CTT TTT GGT-3′ | 8 |
| CPT1C Forward | 5′-TTT CTG GGT GAC GGT GAT CTC-3′ | 9 |
| CPT1C Reverse | 5′-CAT ATG TCC AAT CCC AGT GCA A-3′ | 10 |
| GAPDH Forward | 5′-CAT GAG AAG TAT GAC AAC AGC CT-3′ | 11 |
| GAPDH Reverse | 5′-AGT CCT TCC ACG ATA CCA AAG T-3′ | 12 |
| MT-CO2 Forward | 5′-CCT GCG ACT CCT TGA CGT TG-3′ | 13 |
| MT-CO2 Reverse | 5′-AGC GGT GAA AGT GGT TTG GTT-3′ | 14 |
| ACTB Forward | 5′-TCA CCC ACA CTG TGC CCA TCT ACG A-3′ | 15 |
| ACTB Reverse | 5′-CAG CGG AAC CGC TCA TTG CCA ATG G-3′ | 16 |

MTT proliferation assay: 3000 cells per 96-well were treated for 72 hours using 5 wells per treatment. MTT reagent (M6494, Life Technologies) was prepared as directed, incubated for one hour at 37° C., and analyzed using manufacturer's recommendations.
Statistical analysis: The concordance of stromal CD105 population and epithelial chromogranin A expression was measured with receiver operating characteristic (ROC) curve and the area under the ROC curve (AUC). The p value for AUC (c-statistic) was determined with Mann-Whitney U test. All calculations were performed with ROC package in R. The heat map for neuroendocrine genes was generated by gene signatures under different doses of SFRP1. Clustergram function in bioinformatics toolbox of MATLAB was used for heatmap creation and gene-wise clustering. To pull out the top secreted genes among RNA-sequencing data, ratio values were generated for CAF/CAF^{HiP} and CAF/NAF gene values. Next, the ratio values were ranked for each ratio value among all the genes analyzed, with the highest value having a rank of 1. If there were duplicate ratio values, the average rank was assigned. Subsequently, the ranks of CAF/CAF^{HiP} and CAF/NAF ratio values were summed. The sum values of the two ranks were then ranked. The lowest sum value had the lowest rank, which inversely correlated with the most significant gene expression. As displayed in the heat map, genes with similar patterns were closer to each other in gene expression. cBioPortal was used to check SFRP1, chromagranin A, and CD105 mutation, deletion, and amplification frequency and correlations across publicly available data sets generated by the TCGA Research Network: http://cancergenome.nih.gov/ as described previously (Cerami et al., Cancer Discov. 2012, 2, 401-404; Gao et al., cBioPortal. Sci Signal, 2013, 6, p 11). Multiple comparisons for in vitro data were evaluated with one-way or two-way analysis of variance (ANOVA) using Prism software (GraphPad software) v6.07. The tumor data was analyzed using one-way ANOVA for multiple comparisons. Results were expressed as individual data points or as the mean±S.D. p values of less than 0.05 were considered statistically significant (* p<0.05, ** p<0.01, *** p<0.001, **** p<0.0001). Relative expression within each group of FACS data was plotted with Prism software using the pie or donut chart features.

### Example 3 Antagonizing CD105 Supports Radiation Sensitivity in Prostate Cancer

### CD105 Expression in Prostate Cancer Upon Radiation

CD105 is implicated in aggressiveness, metastasis, recurrence, and resistance to therapy in several cancers including prostate, ovarian, gastric, renal cell, breast, and small cell lung cancer as well as glioblastoma. The inventors found by FACS analysis, that cell surface expression of CD105 on prostate cancer cell lines (PC3, C4-2B, and 22Rv1) increases with 4Gy radiation treatment (FIG. 18A). Expression of cell surface CD105 was both time and radiation dose dependent (FIG. 18B, 18C). While 2Gy radiation did not significantly up regulate CD105 expression, doses of 4Gy and 6Gy significantly increased CD105 for all three cell lines. Further, a time dependent measurement of CD105 expression in 22Rv1 showed a significant elevation by 8 hours post 4Gy radiation, that remained elevated a week later.

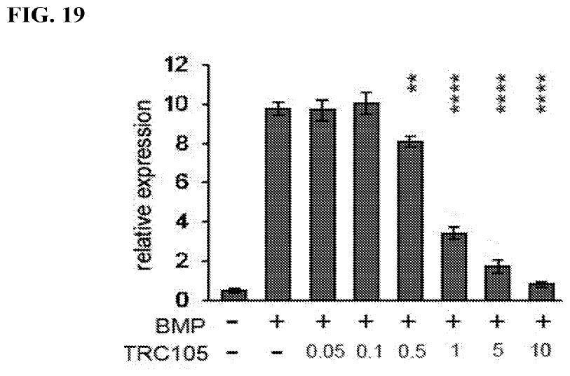

The inventors sought to identify the role of CD105 in radiation response by blocking BMP dependent CD105 signaling using TRC105. At a minimum dose of 1 μg/mL, TRC105 effectively blocked phosphorylated-SMAD⅕ activation and expression of ID1, a BMP target gene, in 22Rv1 when stimulated with 50 ng/mL BMP (FIG. 18D and FIG. 19). Combining TRC105 with radiation significantly increased apoptosis as measured by Annexin-V, compared to radiation alone (FIG. 18E). To determine if CD105 confers radio-resistance, clonogenic survival assays were performed comparing IgG or TRC105 treated CW22Rv1 and C4-2B cell lines with increasing doses of radiation (FIG. 18F). In both these cell lines, treatment with TRC105 sensitized prostate cancer cells to radiation (p value <0.001). Together, radiation induced CD105 seemed to contribute to PCa resistance to apoptosis and antagonizing CD105 with TRC105 restored radiation sensitivity.

### Radiation Induces BMP Mediated SIRT1 Expression

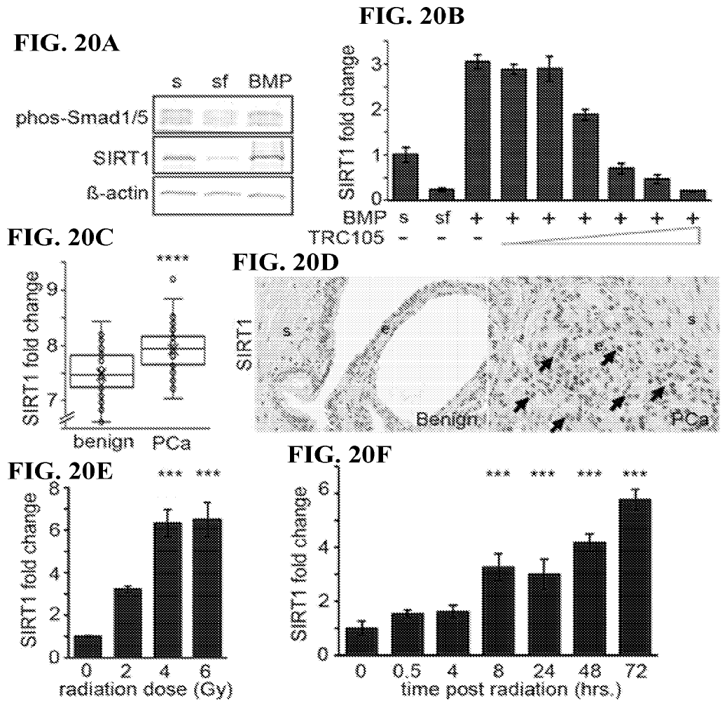

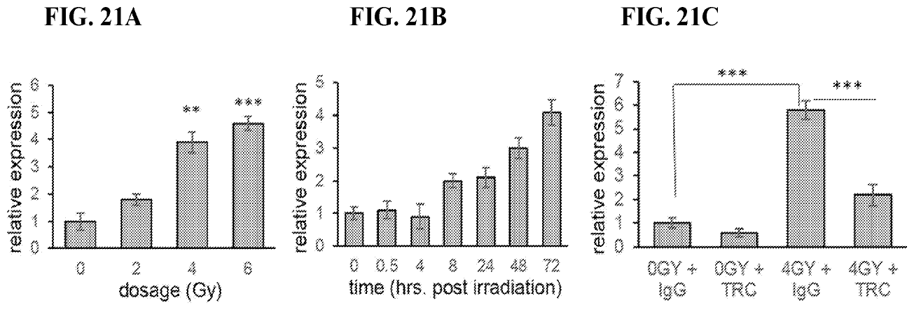

SIRT1, a histone deacetylase, is a well-known mediator of DNA damage repair. The inventors tested if BMP/CD105 signaling regulated SIRT1. Treatment of serum-starved 22Rv1 with BMP induced SIRT1 protein expression associated with phosphorylation of Smad1/5 (FIG. 20A). Further, BMP dependent induction of SIRT1 transcription was down-regulated when CD105 was antagonized with TRC105 in a dose dependent manner (FIG. 20B). SIRT1 has been previously reported to be up-regulated in prostate cancer. Using R2-Genomics analysis, we compared SIRT1 expression in patient samples from benign tissue (n=48) and prostate cancer tissue (n=47). Comparison of tissue types validated SIRT1 expression was significantly overexpressed in prostate cancer samples compared to benign prostate tissue (FIG. 20C). Immuno-staining of human benign and prostate cancer tissue further confirmed SIRT1 is overexpressed in prostate cancer epithelia (FIG. 20D). SIRT1 was immune-localized to the nucleus, as previously reported. As expected, SIRT1 expression was upregulated in both a radiation dose dependent and time dependent manner in 22Rv1 and C4-2B (FIGS. 20E, 20F and 21). Treatment with TRC105 abrogated radiation induced SIRT1 expression in both cell types, which, without being bound to any particular theory, suggests that SIRT1 is downstream of BMP/CD105 signaling.

### Blocking CD105 Induces Transient DNA Damage but Leads to Long Term Accumulation of p53

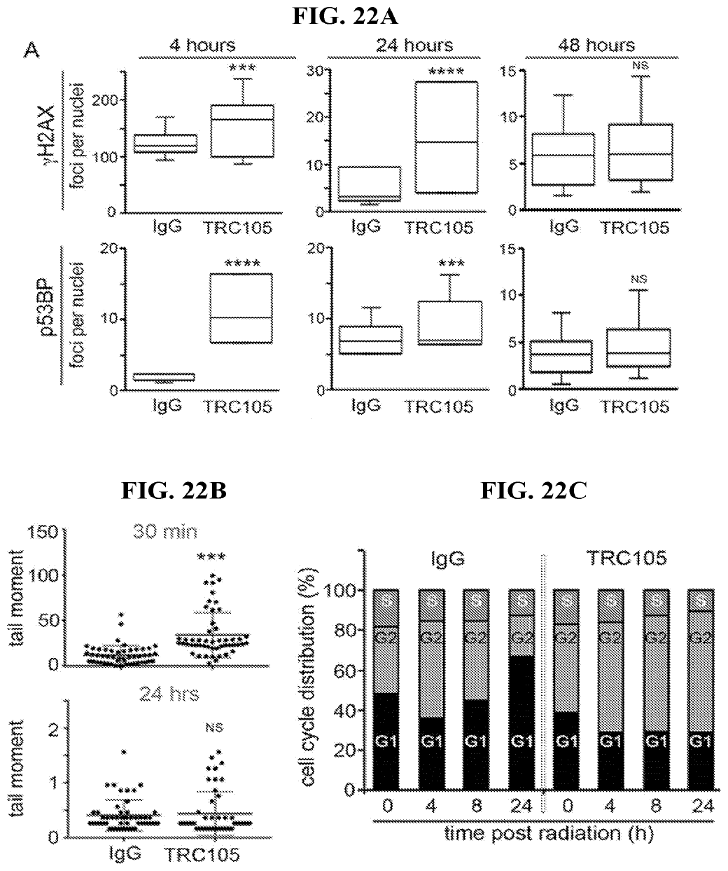

Silencing or knockout of SIRT1 is reported to impair recruitment of downstream DNA damage repair proteins including Nbs1, Brca1, and Rad51. The inventors tested if impairment of DNA damage repair is the mechanism by which TRC105 confers radio-sensitivity, γ-H2AX and p53 binding protein (p53BP) foci were compared at 4, 24, 48, and 72 hours post 4Gy radiation in the presence of IgG or TRC105 (FIG. 22A). While TRC105 treatment resulted in a significant increase in γ-H2AX and p53BP foci at 4 and 24 hours post radiation, by 48 hours there was no difference between TRC105 treated versus radiation alone. TRC105 treatment alone showed a significant increase in double stranded DNA breaks within 24 hours, which persisted past 72 hours, compared to untreated cells (data not shown). However, the number of cells with greater than 10 foci per nuclei was only 4.3% with TRC105, compared to that with radiation alone, 59.6%. To provide a measure of DNA damage, inclusive of the incidence of single stranded breaks, COMET assay was performed with irradiated 22Rv1 cells in the presence and absence of TRC105. The results showed a significant increase in tail moment of TRC105 treated cells 30 minutes post radiation (p value <0.001), but there was no significant difference after 24 hours (FIG. 22B). Without being bound to any particular theory, the data suggest that in the presence of radiation, TRC105 delayed DNA damage repair, however the cells seemed to be able to bypass TRC105-induced SIRT1 repression and restore DNA integrity. Thus, the observed sensitization of prostate cancer cells to radiation with TRC105 was likely not solely determined by its impact on DNA damage repair.

In search for an alternate process mediating TRC105 radiation sensitization the inventors examined changes in cell cycle. The impact of radiation on the cell cycle is well described, as causing a G2 cell cycle arrest that undergoes cell cycle redistribution. Accordingly, the inventors found that irradiating CW22Rv1 cells (4 Gy), in the presence of IgG (control), caused an accumulation of cells in G2 phase by 4 hours, however cell cycle distribution was restored by 8 hours. However, the combination of radiation and TRC105 treated cells underwent G2 cell cycle arrest that did not resolve by 24 hours, despite the observed restoration of DNA integrity in the same timeframe (FIG. 22C). As SIRT1 has previously been reported to regulate p53 stability by de-acetylating p53, the inventors investigated p53 status with TRC105 treatment. 22Rv1 cells were treated with either TRC105 or 200 μM nicotinamide, an inhibitor of SIRT1 activity, prior to irradiation (4Gy). Cells were then collected at 0, 1, and 7 days post radiation to elucidate early and late p53 response. The inventors found that inhibition of SIRT1 activity with nicotinamide or inhibition of SIRT1 expression with TRC105 resulted in an increase in acetylated p53, thereby stabilizing total-p53 (FIG. 22D). Acetylated and total-p53 were markedly increased in TRC105 and nicotinamide treatment groups by 7 days post radiation. Stabilization of p53 with TRC105 or nicotinamide correlated with an increase in p21, a target downstream of p53. Further, p53 stabilization correlated with a decrease in the anti-apoptotic protein Bc1-2. Treating a p53 null prostate cancer cell line, PC3, with TRC105 and increasing doses of radiation for clonogenic survival resulted in no evidence of radiation sensitization. While loss-of-function p53 mutations are rare in prostate cancer, 90% of pancreatic cancers have p53 mutations. We therefore used two p53 mutant pancreatic cancer cell lines: HPAF-II and MIAPACA-2 to identify if TRC105 unresponsiveness to radiation was due to p53 loss of functionality. As with the PC3 cell line, neither the HPAF-II nor MIAPACA-2 cell lines treated with radiation showed a change in clonogenic response with CD105 inhibition by TRC105 (FIGS. 23A-23C). However, two breast cancer cell lines with functional p53, MCF7 and MDAMB231 were found to be positively sensitized to irradiation by the administration of TRC105 (FIGS. 23D and 23E). Without being bound to any particular theory, this suggested that intact p53 response is necessary for TRC105 dependent responsiveness to radiation.

PGC1α and mitochondrial biogenesis are regulated by BMP/CD105

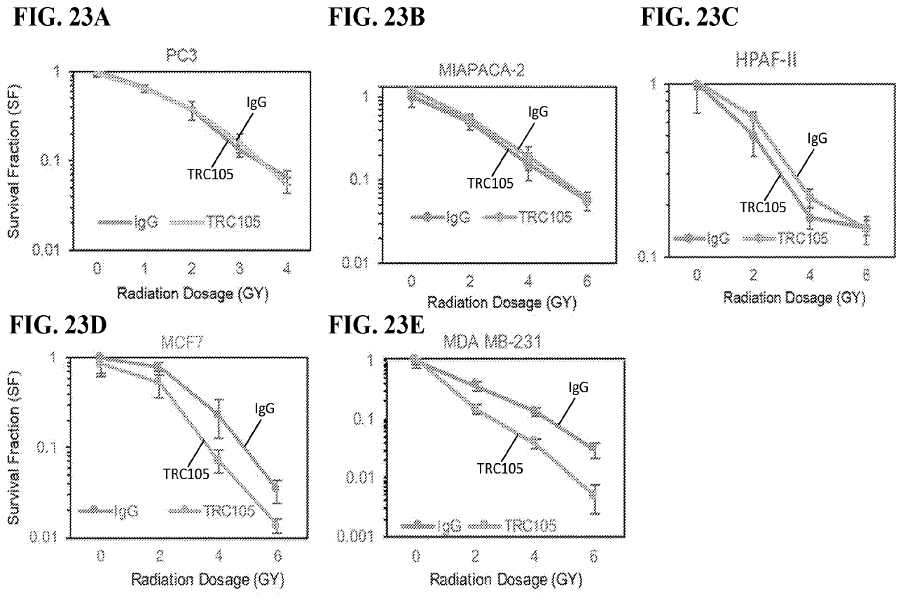

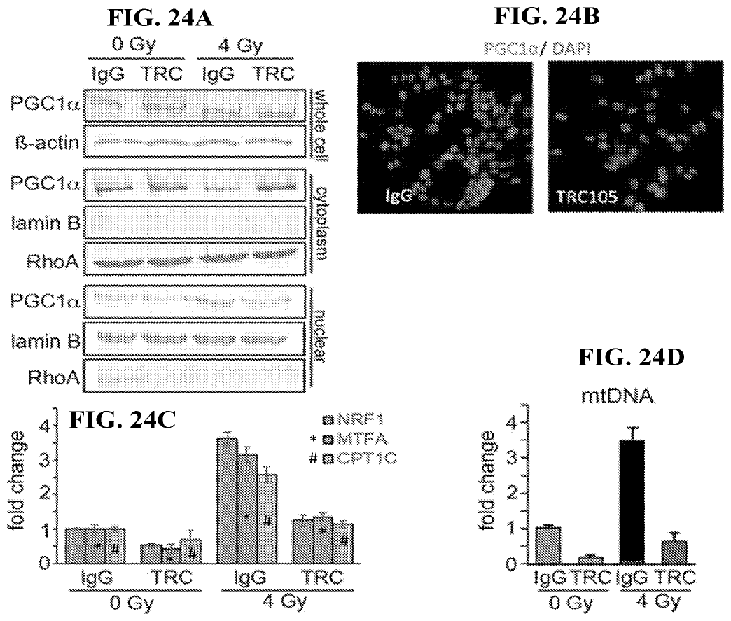

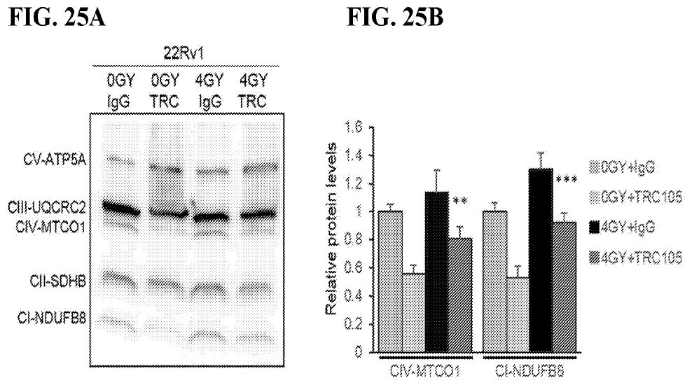

Reexamining the other downstream functions of SIRT1, the inventors tested the role of CD105 on PGC1α. A SIRT1 target, PGC1α is a transcription factor involved in regulating mitochondrial biogenesis. Activation and nuclear localization of PGC1α requires de-acetylation by SIRT1. The treatment of 22Rv1 cells with 4Gy radiation in the presence of IgG or TRC105 had no effect on PGC1α expression, by Western blotting of the whole cell lysate (FIG. 24A). However, closer examination of sub-cellular localization through organelle fractionation, demonstrated PGC1α depletion from the cytoplasmic fraction and accumulation in the nuclear fraction in the context of radiation. Blocking CD105 prevented radiation-induced nuclear translocation of PGC1α. Immunofluorescent localization corroborated these same findings (FIG. 24B). The PGC1α subcellular localization correlated with expression of PGC1α target genes involved in metabolism and mitochondrial biogenesis: NRF1, MTFA, and CPT1C (FIG. 24C). mRNA expression of NRF1, MTFA, and CPT1C were significantly increased with radiation compared to that in the presence of TRC105 (p value <0.001). Radiation has been shown to induce mitochondrial DNA (mtDNA) accumulation in a number of cancer models. Quantitation of mtDNA paralleled the findings thus far with PGC1α regulation by CD105, in that a dramatic down regulation of mtDNA in the presence of TRC105 (p value <0.0001) was found (FIG. 24D). The evaluation of mitochondrial electron transport chain proteins showed TRC105 treatment results in down-regulation of CIV-MTCO1 and CI-NDUF88 (FIG. 25). The inventors demonstrate that CD105 regulation of SIRT1 expression affected both DNA damage repair downstream of p53 as well as maintaining mitochondrial integrity through PGC1α in the context of radiation.

### Antagonizing BMP/CD105 Depletes Cells of Energy

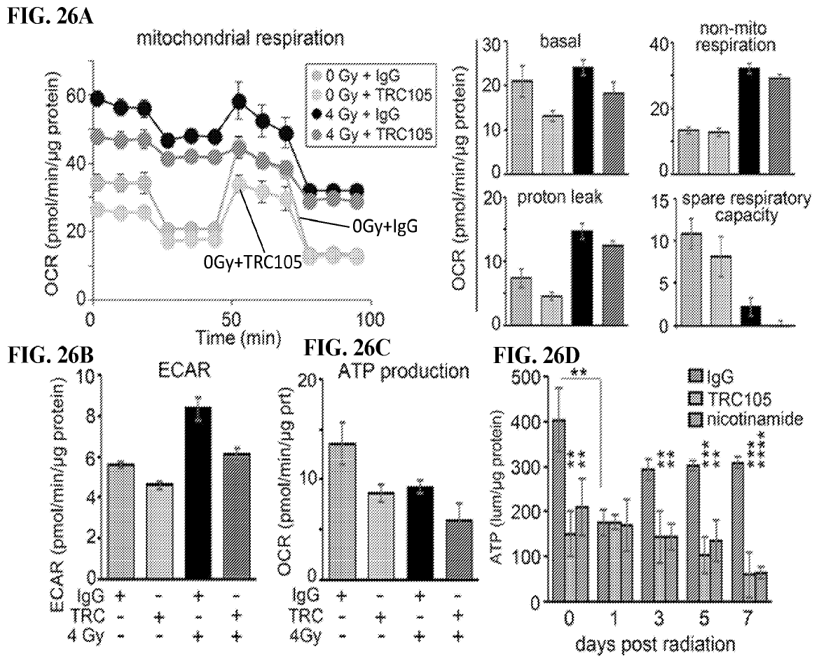

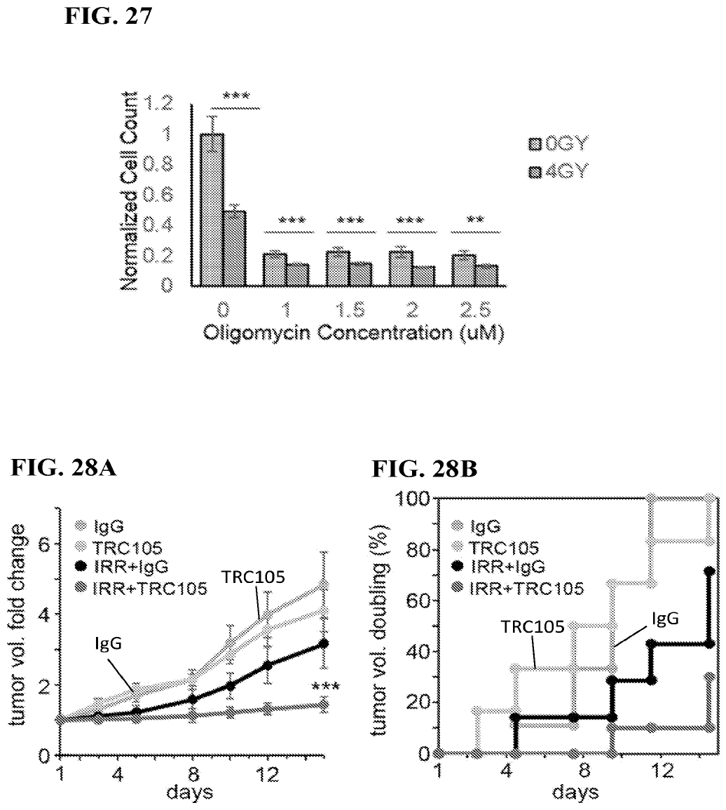

Cell recovery from radiation induced damage requires large amounts of energy and therefore targeting cellular metabolism may sensitize cells to radiation. Previous studies have shown that energy deficits can illicit apoptosis or G2 cell cycle arrest. Prostate cancer is a relatively slow growing cancer that relies heavily on the mitochondria to undergo oxidative phosphorylation. Since CD105 potentiates mitochondrial biogenesis, the inventors studied the functionality of the mitochondria after radiation and TRC105 treatment through the measurement of oxygen consumption rates using Seahorse-XF (FIG. 26A). Radiation treatment elevated non-mitochondrial respiration compared to cells not irradiated. However, when comparing only mitochondrial respiration, the basal oxygen consumption of irradiated to non-irradiated cells was similar. Radiation-mediated mitochondrial damage manifested in increased proton leak and a depletion of spare respiratory capacity. Antagonizing CD105 in the context of radiation resulted in a decrease in basal oxidative phosphorylation and a further decrease in spare respiratory capacity compared to radiation alone. The measure of the extracellular acidification by Seahorse-XF in CW22Rv1 cells suggested a reliance on glycolysis in the context of radiation (FIG. 26B). The addition of TRC105 blocked glycolysis in CW22Rv1 cells. In addition, treatment of either radiation or TRC105 alone caused a depletion of mitochondrial dependent ATP production (FIG. 26C). However, the combination of radiation and TRC105 resulted in a further depletion compared to either agent alone. Accordingly, the treatment of CW22Rv1 with a mitochondrial ATP synthesis inhibitor, oligomycin, significantly reduced cell proliferation, as determined by sequential cell counting, largely irrespective of the dose of oligomycin (p value <0.01, FIG. 27). A significant decrease in total ATP stores was found within 1 day of radiation treatment, that seemed to be restored to levels close to control by 3 days in CW22Rv1 cells (FIG. 26D). When SIRT1 function was blocked directly with nicotinamide or its expression by CD105 antagonism cellular ATP stores were depleted regardless of radiation treatment. Therefore, TRC105 dependent energy depletion is a chronic effect that seems to require a loss of p53 function to enable radio-sensitization.

### Antagonizing CD105 Confers Radio-Sensitivity In Vivo

The inventors assessed CD105 dependent radio-resistance using a CW22Rv1 xenograft model. Mice engrafted with subcutaneous CW22Rv1 were given one dose of IgG or TRC105 72 hours prior to radiation and subsequently 3 times a week for the duration of radiation treatment. The irradiated IgG and irradiated TRC105 groups were given a radiation dosage of 2Gy for 5 consecutive days. Fold change in tumor volume was calculated for each group (FIG. 28A). TRC105 alone did not influence tumor volume compared to untreated. While tumor volumes for radiation and IgG, compared to control, were significantly lower a week after radiation, by 2 weeks this group was not significantly different from the non-irradiated groups. However, the combination of radiation and TRC105 treated tumor volume was significantly lower than the other three experimental groups (p value <0.001). The tumor doubling time was appreciably inhibited by combining TRC105 with irradiation compared to either treatment alone (FIG. 28B).

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method of sensitizing a cancer in a subject in need thereof, comprising:
providing a CD105 antagonist; and
administering the CD105 antagonist to the subject, thereby sensitizing the cancer.

2. The method of claim 1, further comprising administering a cancer therapy.

3. The method of claim 1, further comprising identifying a subject in need of sensitizing a cancer to cancer treatment before administering the CD105 antagonist.

4. The method of claim 1, wherein the cancer is prostate cancer, breast cancer, bladder cancer, lung cancer, colorectal cancer, pancreatic cancer, liver cancer, renal cancer, renal cell carcinoma, melanoma, sarcoma, head and neck, glioblastoma, or a combination thereof.

5. The method of claim 4, wherein the cancer is resistant to radiation and/or androgen targeted therapy.

6. The method of claim 4, wherein the cancer is prostate cancer.

7. The method of claim 1, wherein the CD105 antagonist is an antibody specifically binding to CD105 or an antigen-binding fragment thereof.

8. The method of claim 1, wherein the CD105 antagonist is TRC105 or an antigen-binding fragment thereof.

9. The method of claim 2, wherein the cancer therapy is radiotherapy, chemotherapy, hormone therapy, or surgery, or a combination thereof.

10. The method of claim 2, wherein the subject is treated by the administration of the CD105 antagonist and the cancer therapy.

11. A method of treating, slowing the progression of, reducing the severity of, preventing the recurrence of, and/or reducing the recurrence likelihood of a cancer in a subject in need thereof, comprising:
administering a CD105 antagonist to the subject; and
administering a cancer therapy to the subject, thereby treating, slowing the progression of, reducing the severity of, preventing the recurrence of, and/or reducing the recurrence likelihood of the cancer in the subject.

12. The method of claim 11, wherein the cancer is prostate cancer, breast cancer, bladder cancer, lung cancer, colorectal cancer, pancreatic cancer, liver cancer, renal cancer, renal cell carcinoma, melanoma, sarcoma, head and neck, glioblastoma, or a combination thereof.

13. The method of claim 12, wherein the cancer is resistant to radiation and/or androgen targeted therapy.

14. The method of claim 12, wherein the cancer is prostate cancer.

15. The method of claim 11, wherein the CD105 antagonist is an antibody specifically binding to CD105 or an antigen-binding fragment thereof.

16. The method of claim 11, wherein the CD105 antagonist is TRC105 or an antigen-binding fragment thereof.

17. The method of claim 11, wherein the cancer therapy is radiotherapy, chemotherapy, hormone therapy, or surgery, or a combination thereof.

18. A method of preventing the recurrence of and/or reducing the recurrence likelihood of a cancer in a subject who has been treated with a cancer therapy, comprising:
administering a CD105 antagonist to the subject; and
administering a cancer therapy, thereby preventing the recurrence of and/or reducing the recurrence likelihood of the cancer.

19. The method of claim 18, wherein the cancer is prostate cancer, breast cancer, bladder cancer, lung cancer, colorectal cancer, pancreatic cancer, liver cancer, renal cancer, renal cell carcinoma, melanoma, sarcoma, head and neck, glioblastoma, or a combination thereof.

20. The method of claim 19, wherein the cancer is resistant to radiation and/or androgen targeted therapy.

21. The method of claim 19, wherein the cancer is prostate cancer.

22. The method of claim 18, wherein the CD105 antagonist is an antibody specifically binding to CD105 or an antigen-binding fragment thereof.

23. The method of claim 18, wherein the CD105 antagonist is TRC105 or an antigen-binding fragment thereof.

24. The method of claim 18, wherein the cancer therapy is radiotherapy, chemotherapy, hormone therapy, or surgery, or a combination thereof.
